(19) United States
(12) Patent Application Publication
(10) Pub. No.: US 2026/0007401 A1
(43) Pub. Date: Jan. 8, 2026

(54) SUTURE CONSTRUCT FOR JOINT TISSUE CLOSURE

(71) Applicant: Stryker Corporation, Kalamazoo, MI (US)

(72) Inventors: Charles McCartney, Denver, CO (US); David Marucheau, Denver, CO (US); Greg Broughton, Denver, CO (US); William Kaiser, Campbell, CA (US)

(73) Assignee: Stryker Corporation, Kalamazoo, MI (US)

(21) Appl. No.: 19/253,604

(22) Filed: Jun. 27, 2025

Related U.S. Application Data
(60) Provisional application No. 63/667,195, filed on Jul. 3, 2024.

(51) Int. Cl. A61B 17/04

(52) U.S. Cl. CPC A61B 17/0487; A61B 2017/0464

(57) ABSTRACT

A pre-deployment suture construct includes a working a suture, a tucked suture and a shuttle. The working suture has a first hollow portion and a second hollow portion extending from the first hollow portion, each of the first hollow portion and the second hollow portion being between a fixed tail and a sliding tail of the working suture. The second hollow portion is closer to the sliding tail than the first hollow portion. The tucked suture has a tucked suture loop and a tucked tail extending from the tucked suture loop, at least a portion of the tucked tail being disposed within the second hollow portion of the working suture. Further, the shuttle extends through the first hollow portion and the tucked suture loop.

### CROSS-REFERENCE TO RELATED PATENT APPLICATIONS

This application claims the benefit of and priority to U.S. Provisional Application No. 63/667,195, filed on Jul. 3, 2024, the entire disclosure of which is hereby incorporated by reference herein.

### BACKGROUND

During surgical procedures, the soft tissues around a joint may require securement in order to repair or manipulate the soft tissues. Existing techniques for such repair or manipulation include closure of the soft tissue through knotted and knotless use of sutures. Knot-based techniques introduce a stacked structure comprising one or more knots, which may lead to irritation and adhesions as the knots rub on the tissue. Knots may also be points of failure in suture construction (e.g., forming a weak spot in the suture construction). In knotless techniques, a repair may be completed using a finger trap formed with suture. However, existing knotless approaches are often cumbersome and may be prone to error and/or procedural delay due to the complexity and the significant number of steps required. Further, in many existing methods, undesirably large sutures may be needed to obtain a sought-after repair strength.

Accordingly, a need exists for more effective suture constructs and efficient techniques for closure of soft tissue within joints of the body.

### SUMMARY

In one aspect, the present disclosure relates to a suture construct. In one embodiment, a pre-deployment suture construct includes a working suture including a fixed tail at a first end and a sliding tail at a second end opposite from the first end. The working suture may define a first hollow portion, a second hollow portion, a first entry opening, an exit opening, and a second entry opening. The first hollow portion may extend from the first entry opening to the exit opening, and the second hollow portion is positioned closer to the sliding tail than the first hollow portion. The suture construct may further include a tucked suture including a tucked loop and a tucked tail extending from the tucked loop, where at least a portion of the tucked tail is disposed within the second hollow portion of the working suture via the second opening. The suture construct may also include a shuttle extending through the first hollow portion and the tucked loop, where the sliding tail is configured to be shuttled through the first hollow portion and the tucked loop via the shuttle, and the first hollow portion is configured to provide a finger trap with the sliding tail shuttled through the first hollow portion and the tucked loop.

In one aspect, the present disclosure relates to a method for securing tissue with a suture construct. In an example of an embodiment of the method, the method includes passing one of a fixed tail and a sliding tail of a working suture of a suture construct through or around a tissue, the working suture having a length extending from the fixed tail to the sliding tail. The method may also include engaging the sliding tail of the working suture to a first end of a shuttle, the shuttle including a shuttle tail extending from the first end, a first portion of the shuttle tail extending through a first hollow portion of the working suture and a second portion of the shuttle tail separated from the first end by the first portion passing through a tucked loop of a tucked suture, where a portion of a tucked tail of the tucked suture extends through a second hollow portion of the working suture, and the second hollow portion is further from the fixed tail than the first hollow portion. The method may further include pulling a second end of the shuttle opposite the first end of the shuttle to bring the sliding tail of the working suture through the first hollow portion of the working suture and through the tucked loop of the tucked suture, As described herein, in some embodiments, the first hollow portion provides a knotless fixation region (e.g., a finger trap region, etc.), for example to secure at least a portion of the suture construct. In some embodiments, once the sliding tail is sufficiently tensioned through the pulling step, a knotless fixation region is provided at the first hollow portion (e.g., in the form of a finger trap region, a finger trap, a knotless fixation configuration, etc.) and the tucked loop is cinched.

In one aspect, the present disclosure relates to a kit for securing tissue. The kit may include a working suture including a fixed tail at a first end and a sliding tail at a second end opposite from the first end. The working suture may define a first hollow portion, a second hollow portion, a first entry opening, an exit opening, and a second entry opening, where the first hollow portion extends from the first entry opening to the exit opening, and the second hollow portion is positioned closer to the sliding tail than the first hollow portion. The kit may also include a tucked suture including a tucked loop and a tucked tail extending from the tucked loop, where at least a portion of the tucked tail is disposed within the second hollow portion of the working suture via the second opening. The kit may further include a shuttle extending through the first hollow portion and the tucked loop, and a suture attachment. The suture attachment may be configured to couple at least one of the fixed tail and the sliding tail in a first, pre-deployment configuration and de-couple the at least one of the fixed tail and the sliding tail in a second, deployment configuration, where the sliding tail is configured to be shuttled through the first hollow portion and the tucked loop via the shuttle, and where the first hollow portion is configured to provide a finger trap with the sliding tail shuttled through the first hollow portion and the tucked loop.

This summary is illustrative only and should not be regarded as limiting.

### BRIEF DESCRIPTION OF THE FIGURES

The features, aspects, and advantages of the present disclosure will become better understood with regard to the following description, appended claims, and accompanying drawings in which:

FIG. 1 is a side view of a suture construct in a pre-deployment configuration according to an embodiment of the present disclosure;

FIGS. 2-4 are side views of the suture construct of FIG. 1 in different stages of deployment;

FIG. 5 is a side view of a tucked suture according to an embodiment of the present disclosure;

FIG. 6A is a side view of a suture construct including the tucked suture of FIG. 5 according to an embodiment of the present disclosure;

FIG. 6B is a close up side view of a portion of the suture construct of FIG. 6A;

FIG. 7 is a side view of a tucked suture according to an embodiment of the present disclosure;

FIG. 8 is a cross-sectional view of the tucked suture of FIG. 7;

FIG. 9 is a side view of a suture construct including the tucked suture of FIG. 7 according to an embodiment of the present disclosure;

FIG. 10 is a side view of a suture construct in a pre-deployment configuration according to an embodiment of the present disclosure;

FIG. 11 is a side view of the suture construct of FIG. 10 in a deployed configuration;

FIG. 12 is a side view of a suture construct in a pre-deployment configuration according to an embodiment of the present disclosure;

FIG. 13 is a side view of the suture construct of FIG. 12 in a deployed configuration;

FIGS. 14-16 are side views of deployed suture constructs according to respective embodiments of the present disclosure;

FIGS. 17-18 are side views of a suture construct in different stages of deployment according to an embodiment of the present disclosure;

FIGS. 19-24 are side views of a method of deploying a suture construct according to an embodiment of the present disclosure;

FIG. 25 is a side view of a suture construct according to an embodiment of the present disclosure;

FIG. 26 is a flow chart illustrating a method of using a suture construct to repair a tissue in a patient according to an embodiment of the present disclosure;

FIG. 27A is a top view of a suture construct in a pre-deployment configuration according to an embodiment of the present disclosure;

FIG. 27B is a top view of a portion of a suture construct in a pre-deployment configuration according to an embodiment of the present disclosure;

FIG. 28A is an illustration of the suture construct of FIG. 27A in a stage of deployment according to an embodiment of the present disclosure;

FIG. 28B is an illustration of the suture construct of FIG. 27A in a stage of deployment according to an embodiment of the present disclosure;

FIG. 29 is a top view of the suture construct of FIG. 27A in a stage of deployment according to an embodiment of the present disclosure;

FIG. 30 is a is a top view of the suture construct of FIG. 27A in a stage of deployment according to an embodiment of the present disclosure;

FIG. 31 is a is a top view of the suture construct of FIG. 27A in a stage of deployment according to an embodiment of the present disclosure;

FIG. 32 is a is a top view of the suture construct of FIG. 27A in a stage of deployment according to an embodiment of the present disclosure;

FIG. 33A is a top view of a portion of a process of forming a shuttle loop of the suture construct of FIG. 27A according to an embodiment of the present disclosure;

FIG. 33B is a top view of a portion of a process of forming a shuttle loop of the suture construct of FIG. 27A according to an embodiment of the present disclosure;

FIG. 34A is a top view of a portion of a process of forming a shuttle loop of the suture construct of FIG. 27A according to an embodiment of the present disclosure;

FIG. 34B is a top view of a portion of a process of forming a shuttle loop of the suture construct of FIG. 27A according to an embodiment of the present disclosure;

FIG. 34C is a top view of a portion of a process of forming a shuttle loop of the suture construct of FIG. 27A according to an embodiment of the present disclosure;

FIG. 34D is a top view of a portion of a process of forming a shuttle loop of the suture construct of FIG. 27A according to an embodiment of the present disclosure;

FIG. 34E is a top view of a portion of a process of forming a shuttle loop of the suture construct of FIG. 27A according to an embodiment of the present disclosure;

FIG. 34F is a top view of a portion of a process of forming a shuttle loop of the suture construct of FIG. 27A according to an embodiment of the present disclosure;

FIG. 34G is a top view of a portion of a process of forming a shuttle loop of the suture construct of FIG. 27A according to an embodiment of the present disclosure;

FIG. 34H is a top view of a portion of a process of forming a shuttle loop of the suture construct of FIG. 27A according to an embodiment of the present disclosure;

FIG. 35 is a top view of a process of forming a shuttle loop of the suture construct of FIG. 27A according to an embodiment of the present disclosure;

FIG. 36 is a top view of a process of integrating a shuttle loop and a working suture of the suture construct of FIG. 27A according to an embodiment of the present disclosure;

FIG. 37 is a top view of a process of integrating a shuttle loop and a working suture of the suture construct of FIG. 27A according to an embodiment of the present disclosure;

FIG. 38 is a side view of a tucked loop of the suture construct of FIG. 27A according to an embodiment of the present disclosure;

FIG. 39 is a top view of a process of a tucked strand being integrated with a working suture of the suture construct of FIG. 27A according to an embodiment of the present disclosure;

FIG. 40 is a top view of a process of splicing a tucked strand and a working suture of the suture construct of FIG. 27A according to an embodiment of the present disclosure;

FIG. 41 is a top view of a suture attachment and a working suture of the suture construct of FIG. 27A according to an embodiment of the present disclosure;

FIG. 42 is a top view of a suture attachment and a working suture of the suture construct of FIG. 27A according to an embodiment of the present disclosure;

FIG. 43 is a top view of a suture attachment and a working suture of the suture construct of FIG. 27A according to an embodiment of the present disclosure;

FIG. 44 is a top view of a suture attachment and a working suture of the suture construct of FIG. 27A according to an embodiment of the present disclosure; and

FIG. 45 is a flow chart illustrating a method of using a suture construct to repair a tissue in a patient according to an embodiment of the present disclosure.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

The present disclosure describes various embodiments of suture constructs, associated kits and associated methods intended for use on hard tissue, i.e., bone, or soft tissue. Soft tissue may be, for example, meniscus, cartilage, ligaments and tendons, or the like. While many of the exemplary methods disclosed herein are directed towards use of a suture construct to close a hip capsule, e.g., a tear or opening in a hip capsule, other uses, some of which are described herein, are also envisioned. Exemplary surgical applications include hip repair, shoulder repair, knee repair, repair of other joints in the body, and repair of bone fragments. Specific hip applications include, for example, use in the capsule, labrum, abductor tendons and ligamentum teres. Specific shoulder applications include, for example, use in the rotator cuff, subscapularis, labrum and biceps. Specific knee applications include, for example, use in the meniscus, ACL, PCL and capsule. And, one exemplary application for bone repair may be cerclage to appose two or more bone fragments during healing.

As used herein, “proximal” or “proximally” means closer to or towards an operator, e.g., a surgeon, while “distal” or “distally” means further from or away from the operator. As used herein, the terms “substantially,” “generally,” “approximately,” and “about” are intended to mean that slight deviations from absolute are included within the scope of the term so modified.

The various embodiments disclosed herein generally utilize at least one length of suture. The suture may be a typical suture generally known in the art, and specifically the orthopedic surgery art, such as braided sutures. A braided suture may be formed by braiding or weaving multiple strands together. The strands, in turn, may be formed by combining multiple filaments. In some examples, multiple filaments may be twisted together to form a strand. To avoid ambiguity, such braided sutures may be formed from a series of braided or woven strands, where each strand is formed from a series of filaments. Sutures contemplated by the present disclosure may be substantially hollow or substantially solid depending on the application of the suture included in a particular embodiment. In other words, a suture is at least substantially hollow, as used herein, in that it has some amount of an interior volume along its length in which another suture portion (whether of the same suture or another suture), or other material, may be positioned within the braid; one such example is a braided multifilament suture where the braided strands form an outer sheath surrounding an inner core and/or an interior volume. Alternatively, a solid suture does not include any interior volume within the suture where another suture portion could be positioned; one such example is a monofilament, as well known in the art, while another example may be a flat suture tape which is braided as a flat sheet. The sutures may have a substantially round (e.g., cylindrical) or substantially flat (e.g., tape) shape. Further, stiffness in the suture may be varied. The suture may include one or more ends that are tapered which may provide beneficial uses in terms of passing the smaller suture end through tissue and/or through a body of a suture (or itself) such as in the creation of a splice, providing a knotless fixation region, such as a finger trap, or the like. The suture may be constructed of synthetic material, such as ultra-high molecular weight polyethylene (UHMWPE), polyester (PET), poly lactic-co-glycolic acid (PLGA) or the like, or of organic material (silk, animal tendon, or the like).

Throughout the present disclosure, contemplated suture constructs may include suture portions that are spliced with other suture portions. Examples of splices that may be prepared using the contemplated sutures include brummel splices, tuck splices, sliding loops, eye splices, weaved splices, and/or chain-link splices. The preparation of each of these splices on a suture may be accomplished based on the knowledge of a person having ordinary skill in the art, and by a variety of techniques known in the art, but to avoid ambiguity, some illustrative examples are provided herein. A Brummel splice may be formed by initially taking a first free end of a suture and passing it through a first opening defined by a space between strands of the same suture. Such step may optionally then be followed by taking a second free end of the suture and passing it through a second opening adjacent to the first opening. And, after that, taking the first free end or the second free end and passing it back through a third opening adjacent to the second opening; the other of the first free end and second free end can then be passed back through a fourth opening adjacent at least one of the other openings. These last step(s) may alternatively involve advancing the first free end and/or second free end into the lumen of the suture through the third opening and/or fourth opening, respectively. Once the suture passes are completed, the end or ends are pulled to secure a closed loop. A tuck splice involves taking an end portion of a hollow suture and passing it back into a more proximal region of the same suture via an opening in the suture so that the end portion is advanced through a lumen of the hollow structure in a coaxial fashion.

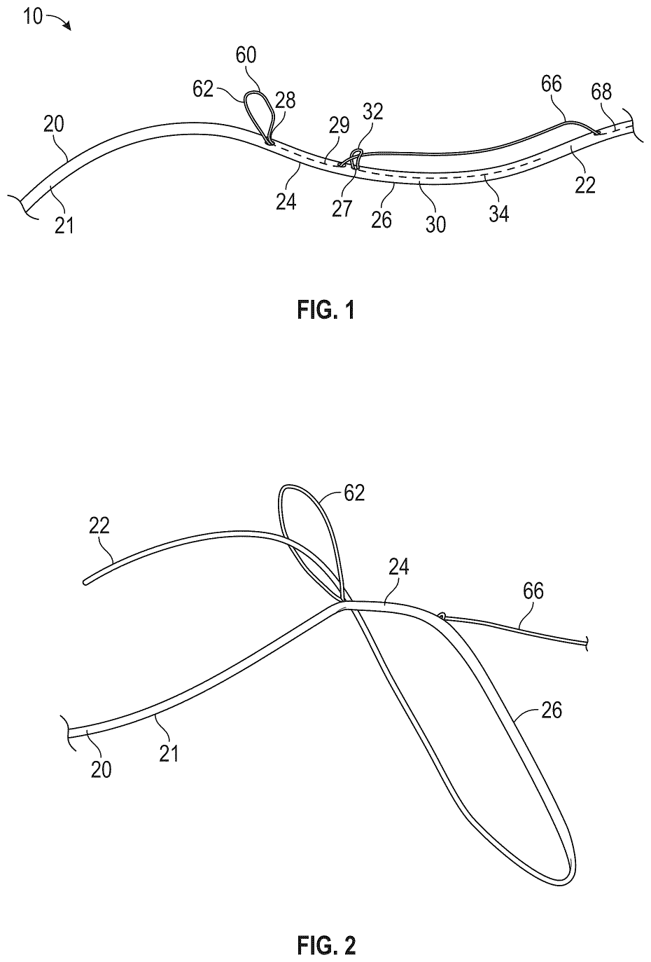

In one aspect, the present disclosure relates to a suture construct for closure of a tissue tear, opening or other gap. In one embodiment, a suture construct 10 is shown in FIGS. 1-4. In a pre-deployment configuration, suture construct 10 includes a working suture 20, a tucked suture 30 and a shuttle 60. When deployed, shuttle 60 is removed from the construct.

Turning initially to the structure of suture construct 10 in the pre-deployment configuration of FIG. 1, working suture 20 includes a fixed tail 21 and a sliding tail 22, with tucked suture 30 and shuttle 60 each disposed therebetween. In some examples, working suture 20 may be a size 2-0, 0, 1 or 2 suture. In other examples, working suture 20 may be a size other than the aforementioned sizes. Further, working suture 20 may have any size suitable for closure of a tissue tear or to repair a bone fracture. Tucked suture 30 includes a tucked loop 32 and a tucked tail 34, where the tucked loop 32 is external to the working suture 20 and the tucked tail 34, extending from the tucked loop 32, passes into a second hollow portion 26 of working suture via tucked suture opening 27 and has a length that continues coaxially within such second hollow portion 26, as shown in FIG. 1, for example. A length of tucked tail 34 may be such that when sliding tail 22 is passed through first hollow portion 24 and tucked loop 32, a portion of tucked tail 34 within sliding tail 22 also passes through first hollow portion 24 and tucked loop 32. In some variations, tucked suture 30 may be prepared so that tucked loop 32 is internal within second hollow portion 26 prior to deployment of suture construct 10. In such variations, sliding tail 22 may be pulled through first hollow portion 24 and tucked loop 32, all internally within working suture 20. In some examples, tucked suture 30 may be a size 2-0 or 0 suture. In other examples, tucked suture 30 may be a size other than the aforementioned sizes. Further, tucked suture 30 may have any size small enough to allow sliding tail 22 to pass coaxially through first hollow portion 24 and also large enough to complement a size of working suture 20. Further, in some examples, tucked loop 32 of tucked suture 30 may be formed via a Brummel splice, a sliding eyelet such as a Munter hitch, and/or any other splice suitable for providing a fixed eyelet. And, in some non-limiting examples, tucked loop 32 may be prepared with an opening size that is in a range from 0.5 mm to 3.0 mm. In still further examples, tucked loop 32 may be a sliding eyelet that closes during tensioning.

Shuttle 60 includes a shuttle loop 62 external to working suture 20 and a shuttle tail 66 that extends from shuttle loop 62 into a first hollow portion 24 of working suture 20 via an entry opening 28 and then exits first hollow portion 24 via an exit opening 29, from which shuttle tail 66 continues through tucked loop 32. A length of working suture 20 between entry opening 28 and exit opening 29, i.e., representative of a knotless fixation region, such as a finger trap region, may be, in some examples, in a range from 10 mm to 40 mm. The length of the first hollow portion 24 may factor into the overall securement strength of the finished suture construct (e.g., with longer knotless fixation regions, or finger traps having higher securement forces). Optionally, a shuttle end portion 68 may be spliced back into working suture 20 in sliding tail 22 of working suture 20, as shown in FIG. 1. In some embodiments, the shuttle end portion 68 is temporarily spliced back into the working suture 20, for example to allow manipulation (e.g., removal, pulling, etc.) of the shuttle end portion 68 from the working suture 20 (e.g., during deployment, etc.). Such arrangement of shuttle 60 may simplify organization of the working suture 20 around a tissue to be operated on prior to deployment of suture construct 10. As to materials, in some examples, shuttle 60 may be made of nitinol wire. In other examples, shuttle 60 may be made of a combination of suture and nitinol wire. In further examples, shuttle 60 may be made of suture only, suture with monofilament, or suture with a portion of monofilament in a loop of shuttle 60. It should be appreciated that although some of the embodiments described herein do not show a shuttle 60, the features of shuttle 60 and the manner of placement of shuttle 60 within working suture 20 may be similarly implemented for such other embodiments as contemplated by the present disclosure.

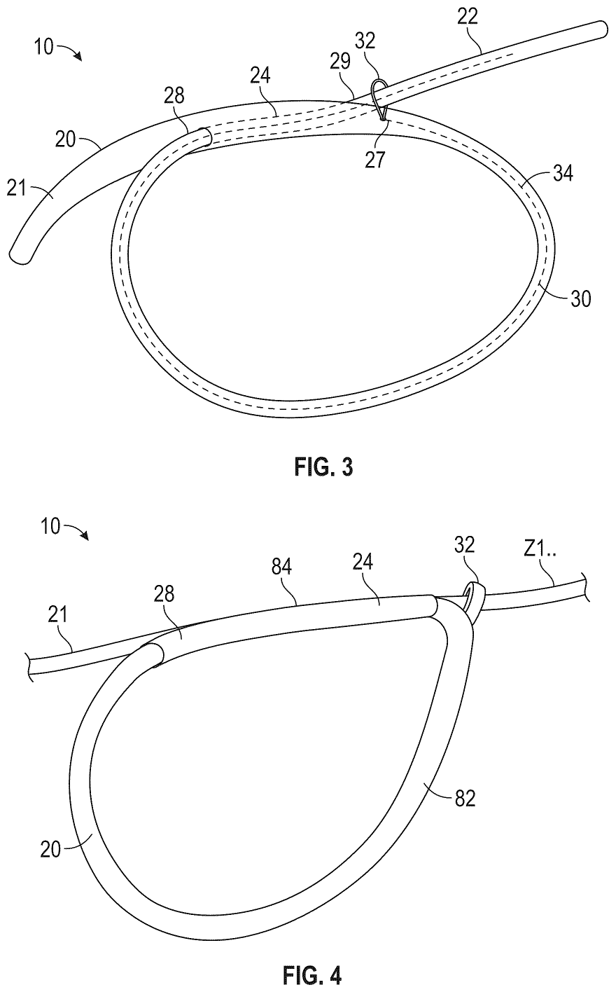

Suture construct 10 is configured to be deployed as shown in the sequence illustrated in FIGS. 1-4. Sliding tail 22 is engaged to shuttle loop 62, as shown in FIG. 2, then shuttle tail 66 is pulled, bringing sliding tail 22 into a lumen of first hollow portion 24 of working suture 20, and, as shuttle tail 66 is pulled farther, out of first hollow portion 24 and through tucked loop 32, as shown in FIG. 3. Sliding tail 22 is pulled sufficiently so that a knotless fixation region or area is provided, shown as a finger trap 84, at the first hollow portion 24, and tucked loop 32 cinches down onto working suture 20, as shown in FIG. 4. A finger trap is a form of knotless fixation via a one-way tensioning mechanism that is characterized by a high-friction engagement created between co-axial suture material. FIG. 4 illustrates a fully deployed suture construct 10. Once finger trap 84 is provided, a portion of suture construct 10 defines a closed loop 82. A diameter of closed loop 82 in the deployed configuration may be, for example, in a range from 5 mm to 15 mm. In other examples, the diameter of closed loop 82 may be outside of this range as a function of the surgical procedure performed and/or the characteristics of the patient anatomy. Further, and as mentioned above, a length of tucked tail 34 may be such that when finger trap 84 is provided, a portion of tucked tail 34 within sliding tail 22 also passes through first hollow portion 24 and tucked loop 32. In some examples, an extent to which tucked tail 34 extends a distance beyond tucked loop 32 is multiple millimeters (e.g., 3.5 millimeters, etc.), or is in a range (e.g., from 1.0 mm to 4.0 mm). In some specific examples, tucked tail 34 extends beyond tucked loop 32 by at least 1.5, 2.0, 2.5, 3.0 or 3.5 mm. In other embodiments, the tucked tail 34 extends beyond the tucked loop 32 another suitable distance (e.g., 1.0, 1.5, 2.0, 2.5, etc. centimeters or inches, etc.) In other embodiments, an end of tucked tail 34 may be closer to tucked loop 32 or on the same side of tucked loop 32 as finger trap 84. The above-described deployment may be incorporated into a method of using and implanting the suture construct, described elsewhere in the present disclosure.

Advantages of suture construct 10 include that tightening of tucked suture 30 against sliding tail 22 of working suture 20 improves performance under cyclic loading and that ultimate tensile strength is increased relative to what would otherwise be obtained solely through a finger trap.

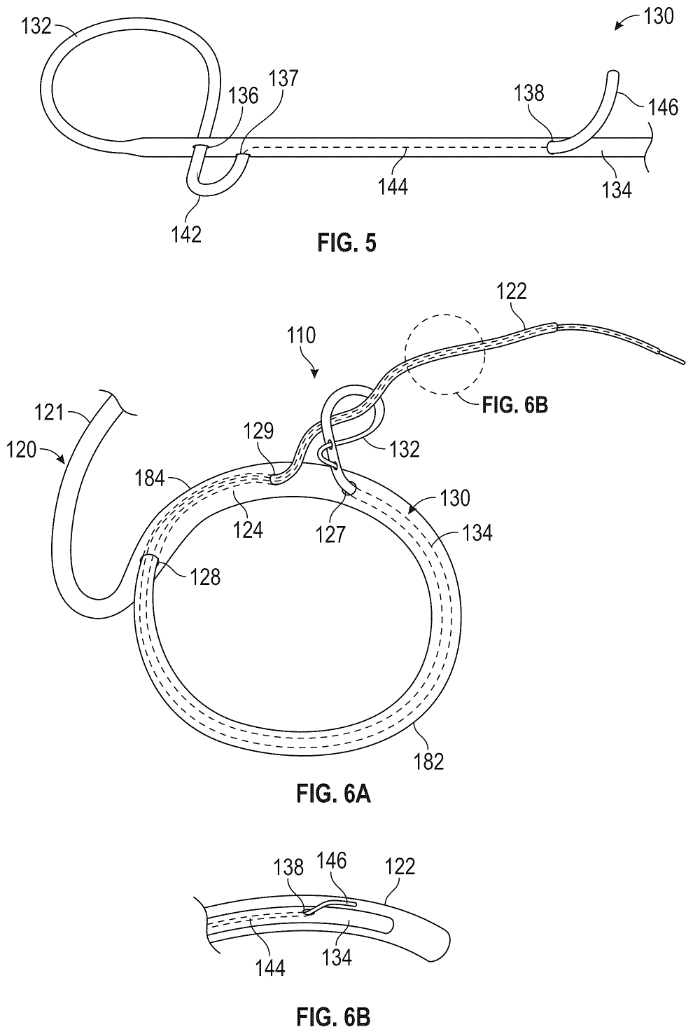

FIGS. 5, 6A and 6B depict another embodiment of a suture construct, indicated by reference numeral 110. In FIGS. 5, 6A and 6B, the 100 series of reference numerals refer to like elements of the double-digit series of reference numerals in suture construct 10 of FIGS. 1-4, unless otherwise noted. In a pre-deployment configuration, suture construct 110 includes a working suture 120, a tucked suture 130 and a shuttle (not shown). When deployed, the shuttle is removed from the construct.

An arrangement of the components of suture construct 110 is the same as suture construct 10 with a difference being the characteristics of tucked suture 130. Accordingly, it should be appreciated that the arrangement of working suture 120, tucked suture 130 and the shuttle in the pre-deployment configuration is the same as that described and shown for suture construct 10. Similarly, deployment of suture construct 110 is accomplished in the same manner as that described for suture construct 10.

One example of tucked suture 130 is shown in FIG. 5 and includes tucked loop 132, tucked tail 134 extending from tucked loop 132, and, on an opposite side of tucked loop 132 from tucked tail 134, a return portion. Specifically, tucked suture 130 is passed through itself at opening 136, then spliced into tucked tail 134 via opening 137 so that tucked suture 130 continues coaxially within a lumen of tucked tail 134 from opening 137 to opening 138, where tucked suture 130 exits the lumen. A portion of tucked suture 130 between openings 136, 137, located external to tucked tail 134, is a first return portion 142. A portion of tucked suture 130 coaxial with the lumen of tucked tail 134 between openings 137, 138 is a second return portion 144, while a final segment of tucked suture 130 external to tucked tail 134 and extending from opening 138 to a free end of the return portion is third return portion 146. This arrangement provides a tucked suture 130 with progressively decreasing bulkiness extending away from tucked loop 132. Specifically, and beginning from tucked loop 132, tucked suture 130 initially includes tucked tail 134 and first return portion 142. Then, further from tucked loop 132, tucked suture 130 includes tucked tail 134 and second return portion 144. Then, still further from tucked loop 132, tucked suture 130 bifurcates and includes tucked tail 134 and third return portion 146, which may be inserted into a working suture in alignment with tucked tail 134, as shown in FIG. 6B. Nonetheless, even when third return portion 146 is in alignment with tucked tail 134, third return portion 146 remains loose relative to tucked tail 134 as it is inclusive of a free end of the return portion. In some examples, the bifurcation may be located approximately 25 mm from a free end of tucked tail 134.

With continued reference to tucked suture 130, in some variations, a length of third return portion 146 may be made shorter than a free end portion of tucked tail 134 measured from opening 138. In this way, another step in decreasing bulk may be making the third return portion 146 shorter than a portion of tucked tail 134 from opening 138 to a free end of tucked tail 134. In a variation, tucked suture 130 may have a return portion that enters tucked tail 134 and extends coaxially within the lumen of tucked tail 134 directly from tucked loop 132. In this manner, the return portion of such variation includes two parts: a portion similar to second return portion 144, and a bifurcated terminal portion similar to third return portion 146. In some examples of the suture construct 110, suture construct 110 may be deployed such that the bifurcation is external to the tucked loop 132 (e.g., while still being located inside of the working suture 120, etc.) after deployment. In other examples, the bifurcation may be proximate to tucked loop 132 after deployment.

The arrangement of tucked suture 130, when used within working suture 120 of suture construct 110, may provide a finger trap 184 easier than constructs with a tucked suture having a uniform tail shape. This is because an initial part of sliding tail 122 passing coaxially through the lumen of first hollow opening 124 is less bulky and only increases in bulk with further passage of the sliding tail 122. Additionally, inclusion of tucked suture 130 with gradations in bulk along its length may improve passage of sliding tail 122 through tucked loop 132 and cinching of tucked loop 132.

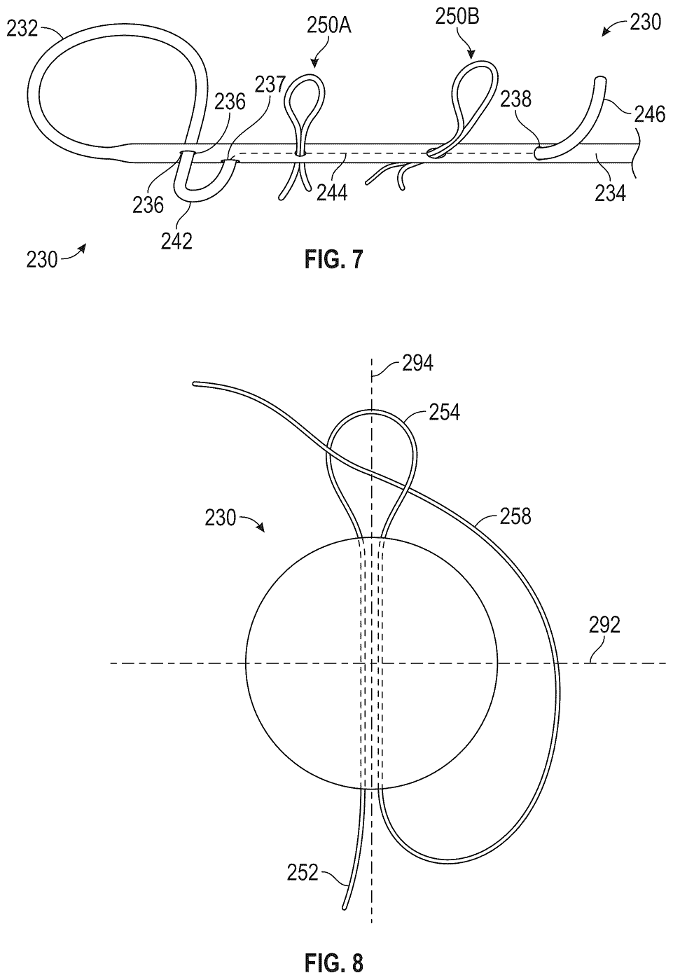

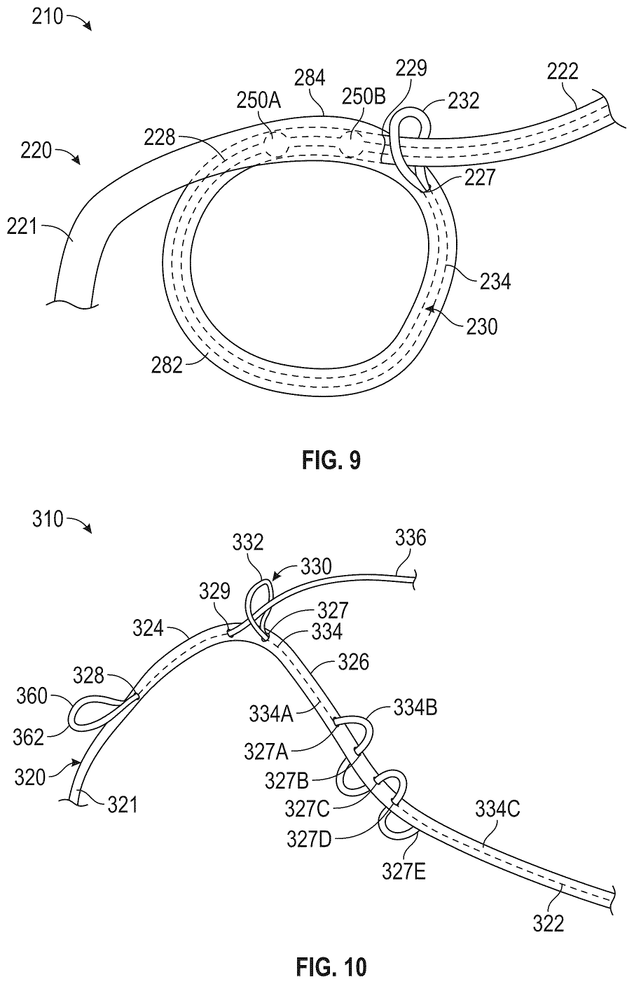

FIGS. 7-9 depict another embodiment of a suture construct, indicated by reference numeral 210 (shown in FIG. 9). In FIGS. 7-9, the 200 series of reference numerals refer to like elements of the double-digit series of reference numerals in suture construct 10 of FIGS. 1-4, unless otherwise noted. In a pre-deployment configuration, suture construct 210 includes a working suture 220, a tucked suture 230 and a shuttle (not shown). When deployed, the shuttle is removed from the construct.

An arrangement of the components of suture construct 210 is the same as suture construct 10 with a difference being the characteristics of tucked suture 230. Accordingly, it should be appreciated that the arrangement of working suture 220, tucked suture 230 and the shuttle in the pre-deployment configuration is the same as that described and shown for suture construct 10. Similarly, deployment of suture construct 210 is accomplished in the same manner as that described for suture construct 10.

As shown in FIGS. 7 and 8, tucked suture 230 includes a tucked loop 232, a tucked tail 234 extending from one side of tucked loop 232, and, from the other side of tucked loop 232, a return portion 242, 244, 246, with the same characteristics as those described and shown for tucked suture 130. A difference with tucked suture 230 is the inclusion of additional standalone sutures that are provided to strategically add bulk, i.e., thickness to the tucked suture 230. Tucked suture 230 may include one or more of such additional sutures, although in the depicted embodiment, two bulk sutures 250A, 250B are included. While first bulk suture 250A is shown in detail in FIG. 8 and described below, it should be appreciated that second bulk suture 250B may include the same features. First bulk suture 250A is passed through tucked suture 230 to increase the bulk of suture at a specific location along a length of tucked tail 234. One end of first bulk suture 250A includes a fixed tail end 252. Fixed tail end 252 may simply be an end portion of a suture that may also be stiffened. In some examples, fixed tail end 252 may include a knot, a spike or other similar gripping component, from which a loop portion 254 of first bulk suture 250A extends transversely through a diameter of tucked suture 230, as shown in FIG. 8. Loop portion 254 exits tucked suture 230 approximately opposite fixed tail end 252 and then reenters tucked suture 230 at a similar location to define a loop outside of tucked suture 230. Loop portion 254 then extends back through tucked suture 230 to once again exit tucked suture 230 at a location generally proximate fixed tail end 252, a tail portion 258 then extending in a loose manner from there, as shown in FIG. 8. The aforementioned description describes an initial placement of first bulk suture 250A. To prepare first bulk suture 250A for use with tucked suture 230 as part of suture construct 210, tail portion 258 is pulled through the loop of loop portion 254. Such step causes fixed tail end 252 to pull against a surface of tucked suture 230 and the loop portion 254 to cinch onto a surface of tucked suture 230 with a section of tail portion 258 therein. At such time, any loose extension of tail portion 258 may be cut and removed. It should further be appreciated that while first bulk suture 250A is shown oriented along y-axis 294 of tucked suture 230 in FIG. 8, that any individual bulk suture may be oriented as desired relative to tucked suture 230. Thus, for example, another bulk suture may be oriented along x-axis 292 of tucked suture 230. Further, an orientation need not conform to a specific axis. In variations of suture construct 210, the bulk suture(s) may be secured using a structure other than loop portion 254. For example, the bulk suture may be secured with a knot, heating techniques or other commonly known joining techniques.

In some examples of the suture construct 210, suture construct 210 may be deployed such that the bulk sutures 250A, 250B are within finger trap 284 after deployment, as shown in FIG. 9. In other examples, the portion of working suture 220 including bulk sutures 250A, 250B may be pulled to a location proximate to or beyond tucked loop 232 after deployment.

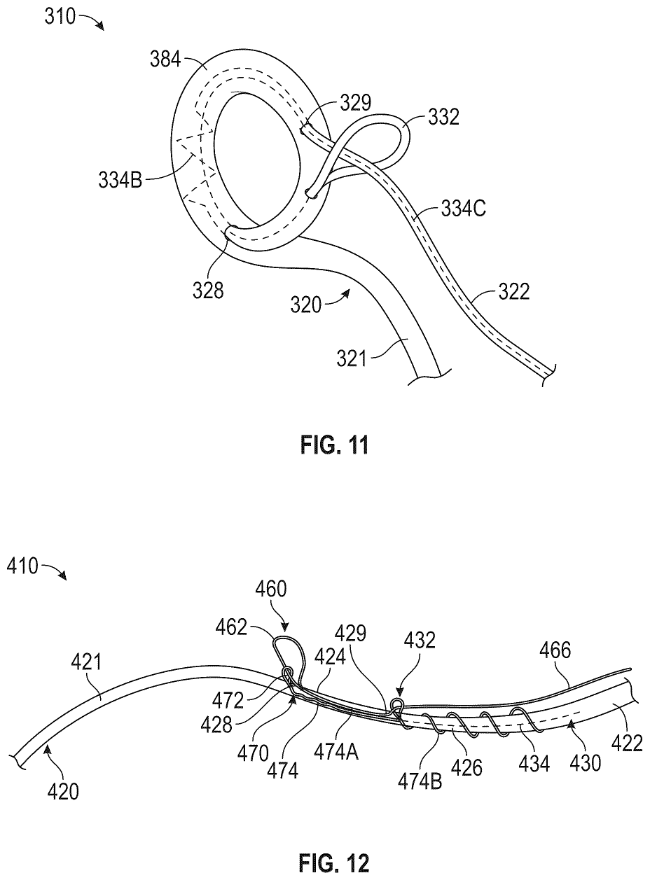

FIGS. 10-11 depict another embodiment of a suture construct, indicated by reference numeral 310. In FIGS. 10-11, the 300 series of reference numerals refer to like elements of the double-digit series of reference numerals in suture construct 10 of FIGS. 1-4, unless otherwise noted. In a pre-deployment configuration, suture construct 310 includes a working suture 320, a tucked suture 330 and a shuttle 360. When deployed, the shuttle 360 is removed from the construct.

Turning initially to the structure of suture construct 310 in the pre-deployment configuration, working suture 320 includes a fixed tail 321 and a sliding tail 322, with tucked suture 330 and shuttle 360 disposed therebetween. Tucked suture 330 includes a tucked loop 332 and a tucked tail 334, where the tucked loop 332 is external to the working suture 320 and the tucked tail 334, extending from the tucked loop 332, includes three separate portions indicative of a changing relationship relative to sliding tail 322 of working suture 320 along a length of sliding tail 322. As shown in FIG. 10, these separate portions of tucked tail 334 include first internal portion 334A, weaved portion 334B, and second internal portion 334C. In a direction moving away from tucked loop 332, first internal portion 334A enters a lumen of second hollow portion 326 via tucked suture opening 327, where first internal portion 334A continues coaxially within the lumen until exiting the lumen at opening 327A. From opening 327A, weaved portion 334B of tucked tail 334 passes transversely and entirely through sliding tail 322 at several spaced apart locations, each being further away from tucked loop 332. As depicted, these locations include openings 327B, 327C, 327D. At opening 327E, tucked tail 334 reenters the lumen of sliding tail 322 and continues coaxially within the lumen of second internal portion 334C to a terminal end of tucked tail 334. In variations, a number of openings in sliding tail 322 through which weaved portion 334B passes entirely through may vary from that shown. The arrangement of tucked suture 330 relative to working suture 320 is advantageous in that when suture construct 310 is deployed, bunching of weaved portion 334B causes tucked loop 332 to be more tightly fastened and provide a stronger grip against sliding tail 322 than it would otherwise have. Further, in some examples, tucked suture 330 may be made of PET to provide an increased grip proximate the finger trap, i.e., via friction, when suture construct 310 is deployed.

Shuttle 360 includes a shuttle loop 362 external to working suture 320 and a shuttle tail 366 that extends from shuttle loop 362 into a first hollow portion 324 of working suture 320 via an entry opening 328 and then exits first hollow portion 324 via an exit opening 329, from which shuttle tail 366 continues and passes through tucked loop 332.

Deployment of suture construct 310 is accomplished in the same manner as that described for suture construct 10, with the deployed suture construct 310 shown in FIG. 11. A location of the weaved portion 334B of tucked tail 334 may be established so that when suture construct 310 is deployed, such weaved portion 334B may be positioned within finger trap 384, as shown in FIG. 11, for example.

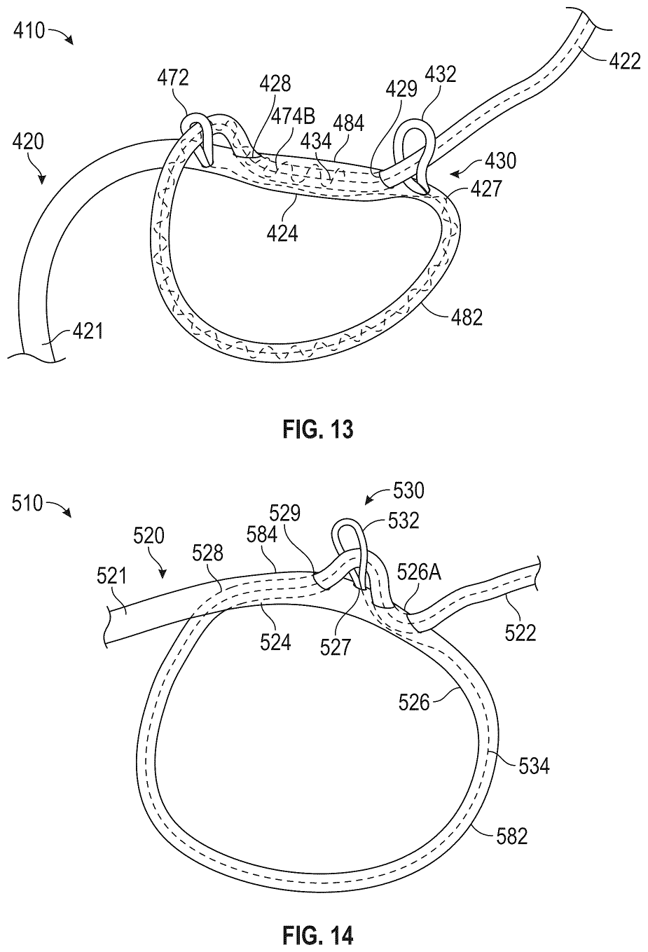

FIGS. 12-13 depict another embodiment of a suture construct, indicated by reference numeral 410. In FIGS. 12-13, the 400 series of reference numerals refer to like elements of the double-digit series of reference numerals in suture construct 10 of FIGS. 1-4, unless otherwise noted. In a pre-deployment configuration, suture construct 410 includes a working suture 420, a first tucked suture 430, a second tucked suture 470 and a shuttle 460. When deployed, the shuttle 460 is removed from the construct.

Turning initially to the structure of suture construct 410 in the pre-deployment configuration, working suture 420 includes a fixed tail 421 and a sliding tail 422. First tucked suture 430 includes a tucked loop 432 and a tucked tail 434 extending therefrom, tucked tail 434 entering a lumen of second hollow portion 426 of working suture 420 and continuing coaxially within the lumen in a direction away from tucked loop 432. Second tucked suture 470 includes a tucked loop 472 and a tucked tail 474 extending therefrom. Tucked tail 474 includes a first portion 474A and a second portion 474B. First portion 474A enters a lumen of first hollow portion 424 of working suture and continues coaxially within the lumen in a direction away from tucked loop 472. Then, at a location in the second hollow portion 426 of working suture 420, second portion 474B of tucked tail 474 exits the lumen and continues toward a free end of sliding tail 422 passing into and out of working suture 420 and/or wrapping around working suture 420, as shown in FIG. 12. In some variations, first tucked suture 430 may enter the lumen of second hollow portion 426 and then exit the lumen and continue toward sliding tail 422 end of working suture 420 passing into and out of working suture 420 and/or wrapping around working suture 420, while second tucked suture 470 enters the lumen of working suture 420 in first hollow portion 424 and extends coaxially within lumen in first and second hollow portions 424, 426 toward sliding tail 422. In some variations, first tucked suture 430 and second tucked suture 470 may be a single suture.

With continued reference to the pre-deployment configuration, shuttle 460 includes shuttle loop 462 and shuttle tail 466 extending from shuttle loop 462, as shown in FIG. 12. Shuttle tail 466 enters the lumen of working suture 420 at entry opening 428 and extends coaxially within the lumen until exiting at exit opening 429. The portion of working suture 420 between respective entry and exit openings 428, 429 is within first hollow portion 424 of working suture 420. A further extent of shuttle tail 466 distal to the segment within the lumen of working suture 420 passes through tucked loop 432, as shown in FIG. 12.

FIG. 13 illustrates a deployed configuration of suture construct 410. Suture construct 410 is configured to form a closure, e.g., used to repair a tear, by passing sliding tail 422 of working suture through tucked loop 472 of second tucked suture 470, then engaging sliding tail 422 with shuttle loop 462. Shuttle tail 466 is then pulled to bring sliding tail 422 into first hollow portion 424 of working suture 420 and through tucked loop 432 of first tucked suture 430. Continued pulling of shuttle 460 provides a knotless fixation region, or knotless fixation (e.g., such as a finger trap, etc.) on working suture 420 between entry and exit openings 428, 429 at the first hollow portion 424, and further causes tucked loops 432, 472 to cinch with working suture 420.

FIGS. 14-16 depict additional embodiments of a suture construct, indicated by reference numerals 510, 610 and 710, respectively. In FIGS. 14-16, the respective 500, 600 and 700 series of reference numerals refer to like elements of the double-digit series of reference numerals in suture construct 10 of FIGS. 1-4, unless otherwise noted. In a pre-deployment configuration, suture construct 510, 610, 710 includes a working suture 520, 620, 720, a tucked suture 530, 630, 730 and a shuttle (not shown). When deployed, the shuttle is removed from the respective suture construct.

As to suture construct 510, illustrated in a deployed configuration in FIG. 14, an arrangement of the components of suture construct 510 is the same as suture construct 10 with a difference being that sliding tail 522, after extending coaxially through first hollow portion 524 within the lumen of working suture 520 and exiting the lumen to pass through tucked loop 532, reenters the lumen of working suture 520 over a distance along a third hollow portion 526A that is within second hollow portion 526, then once again exits the lumen. In some examples, sliding tail 522 passes into a portion of the braid of working suture 520 adjacent to second hollow portion 526 without reentering the lumen, and in still further examples, only partially reenters the lumen. Such arrangement, inclusive of the foregoing examples, is advantageous in that with a finger trap 584 provided, cinching of tucked loop 532 and bunching of such tucked loop 532 with sliding tail 522 may be accomplished and maintained even when sliding tail 522 is cut close to third hollow portion 526A, as the engagement of sliding tail 522 within third hollow portion 526A, i.e., providing a second finger trap, reduces the need for additional suture extending beyond tucked loop 532 in a direction away from finger trap 584. Put another way, the second finger trap laterally adjacent to and/or through third hollow portion 526A enhances the stability of tucked loop 532 and sliding tail 522. Additionally, the second finger trap provides additional clamping when suture construct 510 is deployed. In some examples, third hollow portion 526A may be immediately adjacent to tucked suture opening 527. In other examples, third hollow portion 526A may be proximate but not immediately adjacent to tucked suture opening 527.

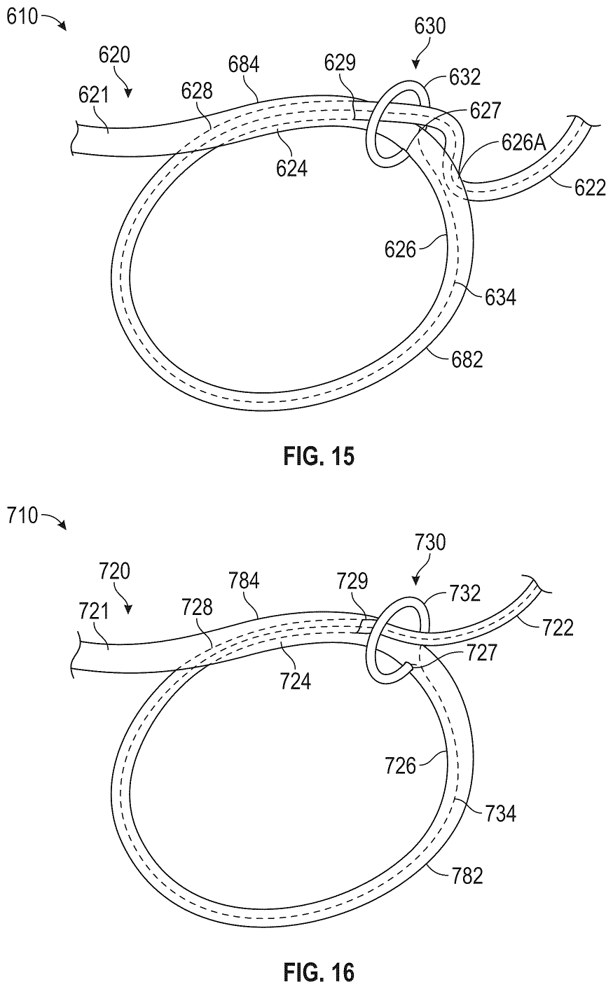

As to suture construct 610, illustrated in a deployed configuration in FIG. 15, an arrangement of the components of suture construct 610 is generally the same as suture construct 10 and further, is the same as suture construct 510 in that it includes a portion of sliding tail 622 extending into or through a lumen of working suture 620 along a third hollow portion 626A (e.g., as shown in at least FIG. 15). A distinction of suture construct 610 compared to suture construct 510 relates to tucked suture 630. In a pre-deployment configuration of suture construct 610, two sides of tucked loop 632 are joined by splice 627 external to working suture 620, with tucked tail 634 entering a lumen of second hollow portion 626. Additionally, a tucked loop 632 of tucked suture 630 is arranged so that two segments of working suture 620 pass through tucked loop 632 when deployed, rather than the tucked loop 632 extending to one side of one segment of the working suture 620 as in suture construct 510. When deployed, as with suture constructs 10, 510, sliding tail 622 is passed through tucked loop 632 and back into or through the working suture 620 (e.g., along the third hollow portion 626A), as shown in FIG. 15. In a variation, splice 627 of tucked loop 632 is located within the lumen in second hollow portion 626 of working suture 620 such that there are two entry locations of tucked loop 632 into working suture 620.

As to suture construct 710, illustrated in a deployed configuration in FIG. 16, an arrangement of the components of suture construct 710 is generally the same as suture construct 10 with a difference being that tucked suture 730 includes a tucked loop 732 positioned relative to working suture 720 so that two segments of working suture 720 pass through tucked loop 732 when deployed. Again, as with the embodiments described above, this includes sliding tail 722 of working suture 720 being passed through tucked loop 732. In a pre-deployment configuration of suture construct 710, two sides of tucked loop 732 are joined by splice 727 external to working suture 720, with tucked tail 734 entering a lumen of second hollow portion 726. Further, in a variation, splice 727 of tucked loop 732 is located within the lumen in second hollow portion 726 of working suture 720 such that there are two entry locations of tucked loop 732 into working suture 720. When deployed, sliding tail 622 is passed through tucked loop 632 and remains outside or external to the working suture 620, as shown in FIG. 16.

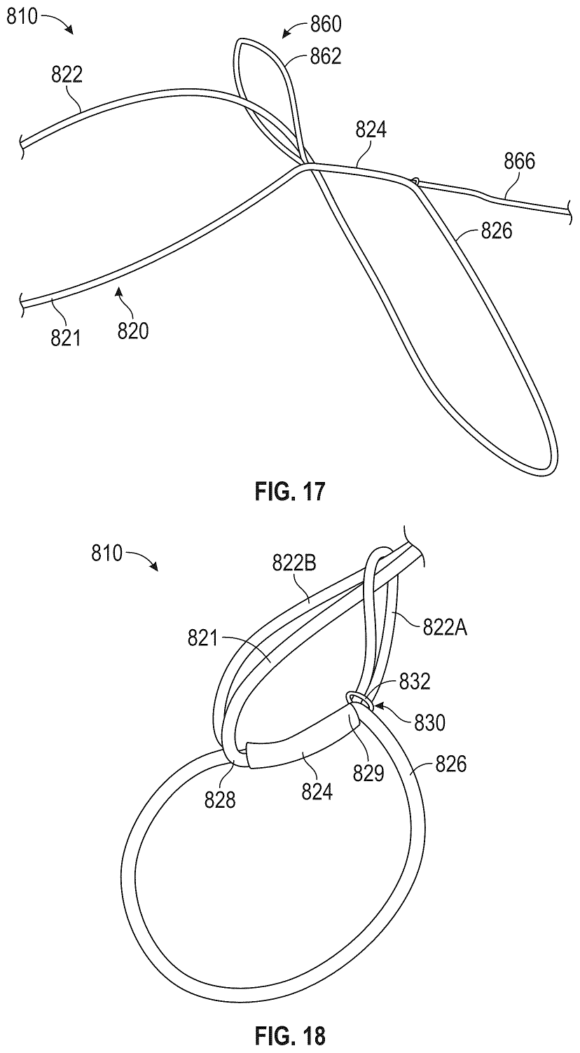

FIGS. 17-18 depict another embodiment of a suture construct, indicated by reference numeral 810. In FIGS. 17-18, the 800 series of reference numerals refer to like elements of the double-digit series of reference numerals in suture construct 10 of FIGS. 1-4, unless otherwise noted. In an exemplary embodiment, the suture construct 810 of FIGS. 17-18 are the same suture construct deployed and/or used in different configurations. In a pre-deployment configuration, suture construct 810 includes a working suture 820, a tucked suture 830 and a shuttle 860. When deployed, the shuttle is removed from the construct.

The sliding tail 822 is passed through the shuttle loop 862. Upon tensioning the shuttle tail 866, the sliding tail 822 is pulled towards and into the first hollow portion 824. This tension on sliding tail 822, with shuttle loop 862 positioned along the length of sliding tail 822, causes the sliding tail 822 to fold onto itself. The shuttle tail 866 is used to pull the sliding tail 822 at least part of the way through the first hollow portion 824, providing a sliding tail closure portion 822A extending out the other end of first hollow portion 824—an example of this intermediate configuration is illustrated in FIG. 18. Both the fixed tail 821 and the sliding tail free end portion 822 are still on the opposite side of the first hollow portion 824 from sliding tail closure portion 822A (e.g., the fixed tail 821 and the sliding tail free end portion 822B have not been pulled into or through the first hollow portion 824). Next, the fixed tail 821 and the sliding tail free end portion 822B are passed through the loop of sliding tail closure portion 822A. FIG. 18 shows the fixed tail 821 and the sliding tail free end portion 822B after they have been passed through the loop of the sliding tail closure portion. Next, the user may pull on the sliding tail free end portion 822B and the fixed tail 821, which causes the loop of sliding tail closure portion 822A to tighten on fixed tail 821 and the sliding tail free end portion 822B. The tightening of the loop of sliding tail closure portion 822A on both the fixed tail 821 and the sliding tail free end portion 822B functions to secure the suture construct 810 when deployed.

In some embodiments, the sliding tail 822 is folded over the shuttle loop 862. The shuttle tail 866 is used to pull the sliding tail 822 part of the way through the first hollow portion 824. As shown in FIG. 18, part of the folded over portion of the sliding tail 822 is pulled through the first hollow portion 824 via the shuttle. In FIG. 18, a loop of sliding tail closure portion 822A is shown. Both the fixed tail 821 and the sliding tail free end portion 822 are still on the opposite side of the first hollow portion 824. (i.e., the fixed tail 821 and the sliding tail free end portion 822B have not been pulled all the way through the first hollow portion.) Next, the fixed tail 821 and the sliding tail free end portion 822B are passed through the loop of sliding tail closure portion 822A. FIG. 18 shows the fixed tail 821 and the sliding tail free end portion 822B after they have been passed through the loop of the sliding tail closure portion. Next, the user would pull on the sliding tail free end portion 822B and the fixed tail 821, which causes the loop of sliding tail closure portion 822A tighten on fixed tail 821 and the sliding tail free end portion 822B. The tightening of the loop of sliding tail closure portion 822A on both the fixed tail 821 and the sliding tail free end portion 822B functions to secure the suture construct 810 when deployed.

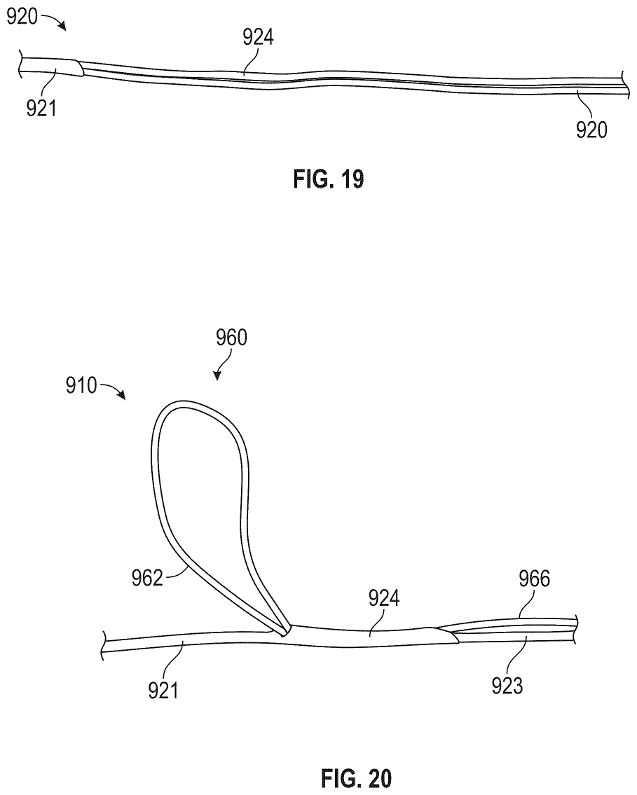

FIGS. 19-24 depict another embodiment of a suture construct, indicated by reference numeral 910. In FIGS. 19-24, the 900 series of reference numerals refer to like elements of the double-digit series of reference numerals in suture construct 10 of FIGS. 1-4, unless otherwise noted. In a pre-deployment configuration, suture construct 910 includes a working suture 920 and a shuttle 960. When deployed, the shuttle is removed from the construct.

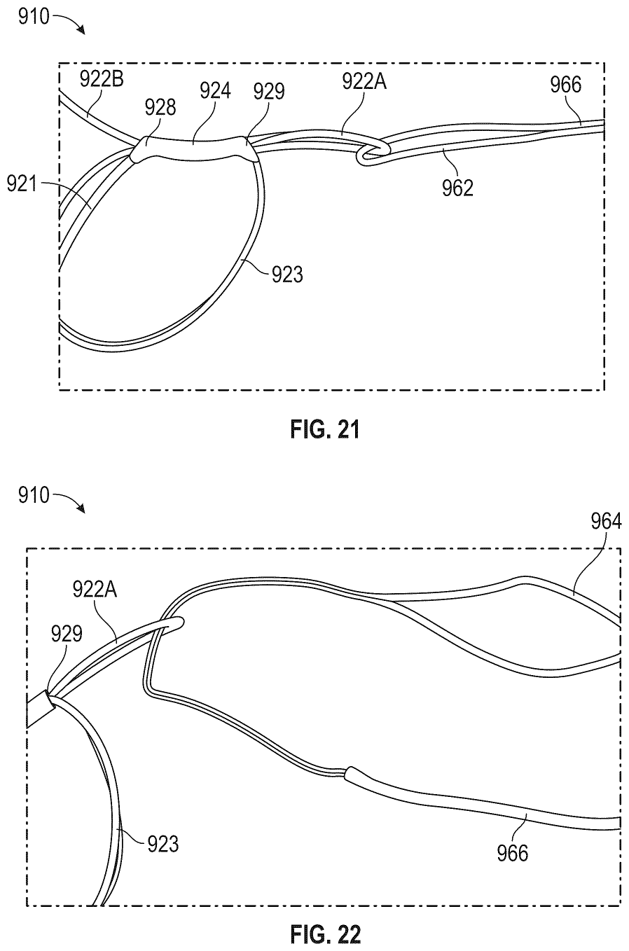

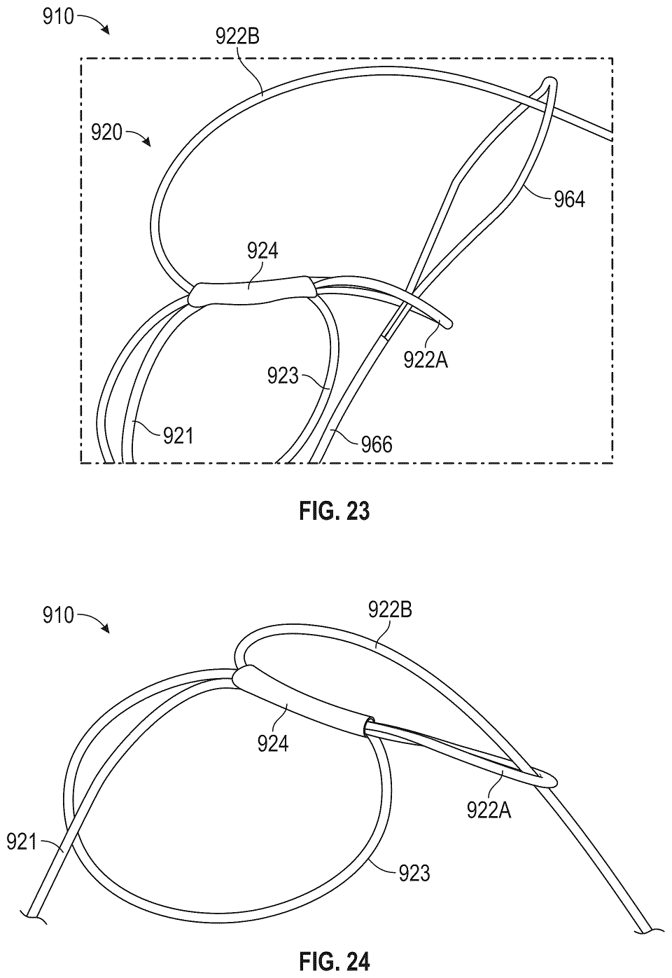

An arrangement of the components of suture construct 910 is the same as suture construct 810 with a notable difference being in the characteristics of the working suture. Specifically, working suture 920 includes a portion that is round and hollow, and another portion that is a flat suture, also known as “suture tape.” Such flat portion is indicated by reference numeral 923 in FIGS. 19-24. In some examples, flat portion 923 may be spliced with hollow portion 924 to combine the two parts into a single suture. During deployment, hollow portion 924 and flat portion 923 of working suture 920 are positioned as follows. Sliding tail 922, inclusive of flat portion 923, is folded over shuttle loop 962, and, in the same manner as described above for suture construct 810, is pulled into the lumen of working suture 920 coaxially through first hollow portion 924, as shown in FIG. 21. The portion of working suture 920 that is pulled through first hollow portion 924 is all part of flat portion 923. A free end of sliding tail free end portion 922B, also part of flat portion 923, may then be passed through the loop of sliding tail closure portion 922A, as shown in FIGS. 23-24, to cinch sliding tail closure portion 922A onto a remainder of working suture 920. While a tucked suture is not depicted in FIGS. 19-24, it is contemplated that variations of suture construct 910 may include a tucked suture. In one example, a tucked loop of a tucked suture may be weaved into flat portion 923. In another example, flat portion 923 may include a window or opening to accommodate passage of a tucked suture. In still further examples, other means may be used to engage a tucked suture with flat portion 923.

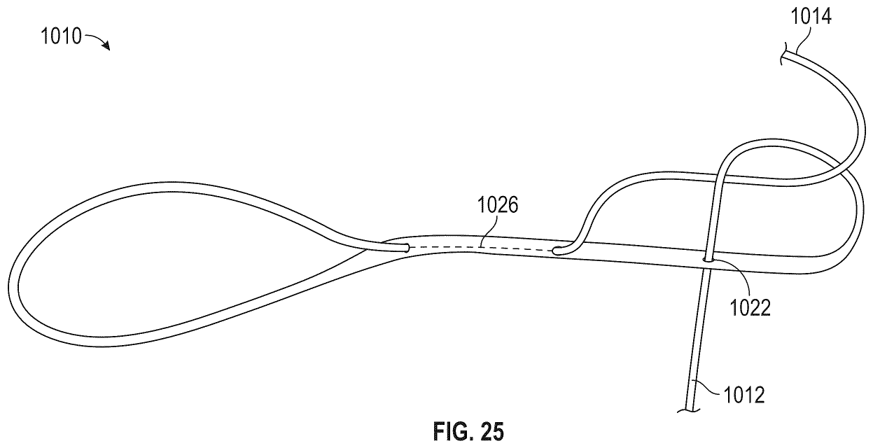

FIG. 25 depicts another embodiment of suture construct, indicated by reference numeral 1010. Suture construct 1010 is a self-splice tucked loop and includes a single suture 1010 extending from a first end 1012 to a second end 1014. Second end 1014 of suture 1010 is passed into another part of suture 1010 through hollow portion 1026, and, closer to first end 1012, first end 1012 is transversely passed transversely and entirely through an opening 1022 in suture 1010. First and second ends 1012, 1014 may be tensioned as needed to form a secure loop.

In another aspect, the present disclosure relates to a kit for preparing a closure of soft tissue. It is contemplated that the kit may include suture and other structures as contemplated by the present disclosure. In one embodiment, a kit may include a working suture. In one embodiment, a kit may include a working suture and a shuttle. In some embodiments, a kit may include two or more working sutures and/or two or more shuttles. In some examples of the aforementioned embodiments, one or more of the working sutures may include a tucked suture partially positioned through a lumen of the working suture. In other examples of the aforementioned embodiments, one or more of the working sutures may include a hollow portion and a flat portion. In any one of the contemplated embodiments, a kit may include any number of accessories used to deploy a suture construct. For example, a needle may be included in a kit. The contents of the kit may be provided in a single package or in a series of packages. In some instances, each component of a kit may be provided in a separate package. A kit may further be provided with an instruction manual with details on how to use the contents of the kit. In some embodiments, instructions may be printed on the packaging itself. In any one of the embodiments that includes packaging, the packaging itself may include indicia and/or surfaces shaped to organize and/or label different components of a kit. In one specific example of a suture construct kit indicated for hip repair and including multiple suture strands, the packaging of the kit may include features to organize the suture strands so that a user knows which strand to tension first, second, and so on.

In another aspect, the present disclosure relates to a method of using a suture construct to close or seal a soft tissue. Such closure may be for the purpose of closing a tear to prevent fluid from being lost from a fluid enclosure in the body. One exemplary location in the body to perform such method is on the hip capsule. For the sake of brevity, the methods described below refer to the hip capsule, though as explained elsewhere in the present disclosure, the method may be employed in many other areas of the body.

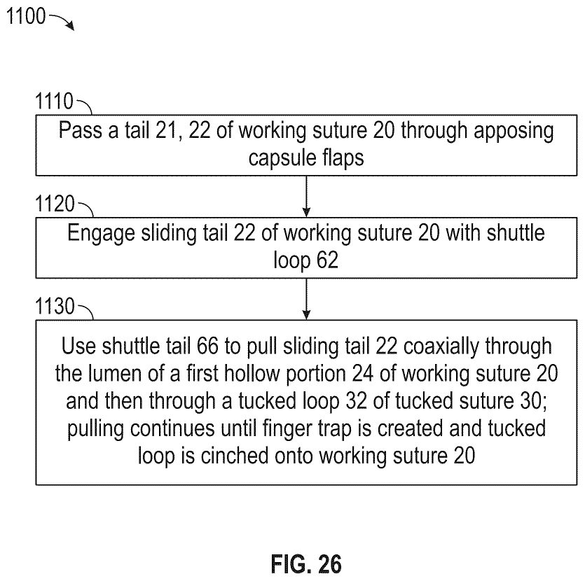

In one embodiment, suture construct 10 is used to repair a torn hip capsule in a patient, a method shown in FIG. 26. After access to the hip capsule of the patient is created, a step 1110 includes passage of fixed tail 21 or sliding tail 22 of working suture 20 through one side of the tear and then out of an opposing side, e.g., through apposing capsule flaps. Then, in step 1120, sliding tail 22 is engaged with shuttle loop 62. In step 1130, shuttle tail 66 is used to pull sliding tail 22 coaxially through the lumen of first hollow portion 24 of working suture 20 and then through tucked loop 32 to provide a finger trap and to cinch tucked loop 32 onto working suture 20 in a single step. If needed, sliding tail 22 may be pulled further to tighten the repair. Fixed tail 21 may be used to provide tension during performance of method steps. At this juncture, the tear of the hip capsule is closed and sealed without the need for any further steps. Extra suture on sliding tail 22 and fixed tail 21 may be cut as desired and appropriate steps may be taken to close the access to the patient. The above-described method may be similarly performed using any one of suture constructs 110, 210, 310, 510, 610, 710.

In another embodiment, suture construct 410 is used to repair a torn hip capsule in a patient. The method using suture construct 410 may be performed in generally the same manner as the method utilizing suture construct 10 except that prior to engaging sliding tail 422 onto shuttle loop 462, sliding tail is passed through tucked loop 472.

In yet another embodiment, suture construct 810 is used to repair a torn hip capsule in a patient. The method using suture construct 810 may be performed in generally the same manner as the method utilizing suture construct 10 except that when shuttle tail 866 is used to bring sliding tail 822 coaxially through first hollow portion 824 and through tucked loop 832, a sliding tail closure portion 822A remains a loop after pulling with shuttle tail 866 is completed. At this juncture, a free end of sliding tail free end portion 822B may be passed through the loop of sliding tail closure portion 822A to cinch the loop and complete the closure of the tear of the hip capsule.

In yet another embodiment, suture construct 910 is used to repair a torn hip capsule in a patient. The method using suture construct 910 may be performed in generally the same manner as the method utilizing suture construct 810. As depicted in FIGS. 19-24, the method of using suture construct 910 includes usage of a shuttle 960 as follows. From a configuration as shown in FIG. 20, sliding tail 922 is engaged to a shuttle loop 962. Then, as shown in FIG. 21, shuttle tail 966 is pulled to bring a folded over portion of sliding tail 922 through first hollow portion 924, such portion becoming sliding tail closure portion 922A. At this juncture, a splice that defines shuttle loop 962 is undone by pulling out or cutting a portion of shuttle loop 962 spliced within the lumen of shuttle tail 966, such portion itself being a secondary shuttle loop 964, as shown in FIG. 22. Then, sliding tail free end portion 922B is passed through secondary shuttle loop 964, as shown in FIG. 23, and shuttle tail 966 is pulled to bring sliding tail free end portion 922B through the loop of sliding tail closure portion 922A, with continued pulling causing cinching of working suture 920. In certain variations of suture construct 910 that do not include a tucked loop, sliding tail closure portion 922A need not pass through a tucked loop after being pulled coaxially through first hollow portion 924 of working suture 920. In another embodiment, the above-described usage of shuttle 960 may be similarly employed using suture construct 810.

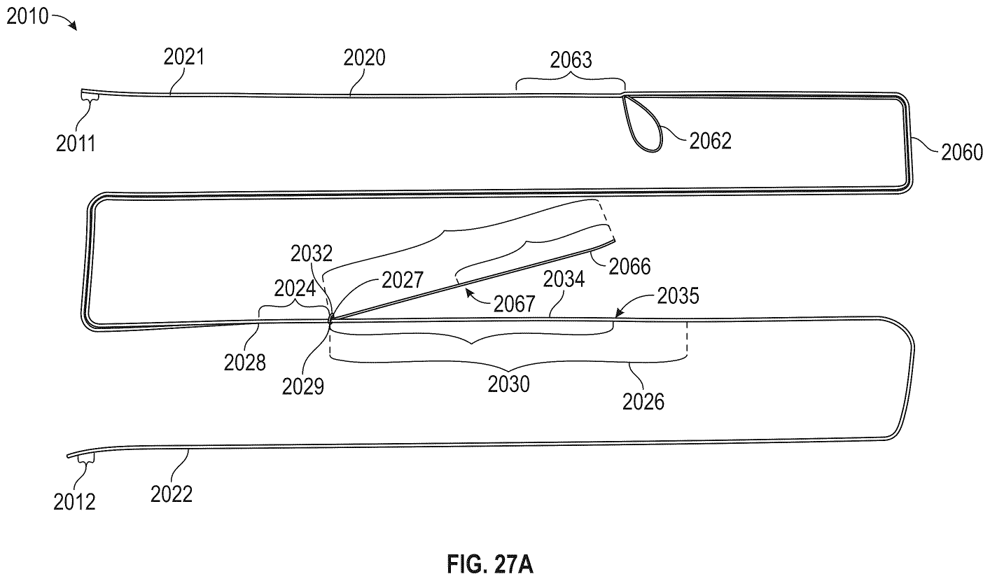

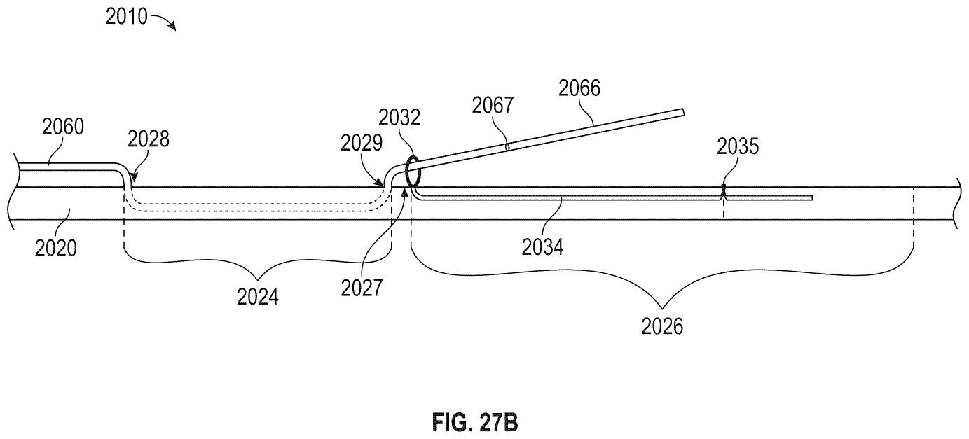

Referring generally to FIGS. 27A-45, a suture construct, a method of forming the suture construct, and a method of using the suture construct for closure of a tissue tear or other gap is shown, according to an example embodiment. The suture construct of FIGS. 27A-44, shown as suture construct 2010, may include the same or similar features, elements, and/or components of the suture construct(s) of FIGS. 1-26, as described herein. In an exemplary embodiment, the suture construct 2010 is movable (e.g., manipulated, etc.) between one or more configurations. For example, the suture construct 2010 may be provided in a first, pre-deployment configuration, and movable to a second, or deployed configuration. In some embodiments, the suture construct 2010 may be provided in one or more deployed configurations. As will be discussed herein, it is contemplated that the suture construct 2010 may be movable (e.g., manipulated, etc.) between one or more deployed configurations, for example in sequence and to provide the suture construct 2010 in a deployed configuration. In some embodiments, the suture construct 2010 may be otherwise movable to one or more deployed configurations.

As shown, the suture construct 2010 includes a first suture, shown as a working suture 2020, a second suture, shown as a tucked suture 2030, and third suture, shown as a shuttle 2060. In an example embodiment, in a second or deployed configuration, the shuttle 2060 is removed from the suture construct 2010.

As shown, the working suture 2020 includes a first end, portion, or tail, shown as fixed tail 2021, and a second end, portion, or tail, shown as sliding tail 2022. Similar to other variations discussed above, the tucked suture 2030 and/or the shuttle 2060 may be disposed between the fixed tail 2021 and the sliding tail 2022. In an example embodiment, and in a first configuration or a pre-deployment configuration, the working suture 2020 defines a length (e.g., between ends of the fixed tail 2021 and the sliding tail 2022). For example, according to an exemplary embodiment the working suture 2020 defines a length of approximately 46 inches between ends of the fixed tail 2021 and the sliding tail 2022. In other embodiments, the working suture 2020 defines another suitable length in the pre-deployment configuration (e.g., approximately 35, 40, 45, 50, 55, 60, etc. inches, etc.).

The working suture 2020 is shown to include a first end or tip, shown as fixed tip 2011, and a second end or tip, shown as sliding tip 2012. The fixed tip 2011 may be positioned at an end of the fixed tail 2021, and the sliding tip 2012 may be positioned at an end of the sliding tail 2022. According to an exemplary embodiment, the working suture 2020 (e.g., the fixed tail 2021, the sliding tail 2022, the portions between ends of the fixed tail 2021 and the sliding tail 2022, etc.) is a size 0 suture. However, in other embodiments, one or more components of the working suture 2020 (e.g., the fixed tail 2021, the sliding tail 2022, etc.) is/are another suitable size (e.g., a 3-0, 2-0, 1, 2, etc. sized suture). In some embodiments, and as will be described herein, the suture construct 2010 may include and/or may be configured to removably couple one or more attachment components (e.g., attachable loops, attachable or spliced sutures, shuttles, bulking features, etc.). For example, the suture construct 2010 may include, and/or the working suture 2020 may be configured to removably couple, one or more attachment components to facilitate manipulation of the suture construct 2010 for closure of a tissue tear or to repair a bone fracture, as described herein. In one example, working suture 2020 may include a leading portion, associated with fixed tip 2011, and/or a leading portion, associated with sliding tip 2012 which may be larger, smaller or the same diameter, shape, denier and/or materials as working suture 2020. For example, one or both such leading portions may be more readily associated with an instrument, such as a suture passer for passing working suture 2020 though tissue. For example, one or more of the leading portions may be stiffer than other portions of the working suture 2020 to aid in loading of the suture construct 2010 into an instrument (e.g., a suture passer, etc.).

According to an exemplary embodiment, the working suture 2020 is a coreless suture. For example, the working suture 2020 may be completely hollow (e.g., between ends of the fixed tail 2021 and the sliding tail 2022, between the fixed tip 2011 and the sliding tip 2012, etc.). As will be described herein, in some embodiments the working suture 2020 includes one or more hollow portions (e.g., a first hollow portion, a second hollow portion, etc.), which may be positioned along at least one or more portions of a length of the working suture 2020. In some embodiments, the hollow portions (e.g., a first hollow portion and a second hollow portion, etc.) are part of the same hollow portion or length of the working suture 2020. In other embodiments, the hollow portions (e.g., a first hollow portion and a second hollow portion, etc.) are part of different hollow portions and/or lengths of the working suture 2020, for example separated by a distance or length, another hollow portion or core, and/or by differences in characteristics of the working suture 2020 (e.g., differences in braiding structure of the working suture 2020, different in diameter or hollow characteristic of the working suture 2020, etc.). In yet other embodiments, the working suture 2020 is a cored suture, for example containing one or more core portions, positioned at or along one or more portions of a length of the working suture 2020, and/or otherwise positioned relative to components or features of the working suture 2020, as described herein. All such embodiments are contemplated herein.

As shown, the working suture 2020 includes a first portion or hollow portion, shown as first hollow portion 2024, and a second portion or hollow portion, shown as second hollow portion 2026. According to an exemplary embodiment, the first hollow portion 2024 and/or the second hollow portion 2026 provide a knotless fixation region. For example, the first hollow portion 2024 and/or the second hollow portion 2026 may provide a region that provides knotless fixation (e.g., via a structural, material, and/or configuration of the first hollow portion 2024 and/or the second hollow portion 2026, etc.). In some embodiments, the first hollow portion 2024 and/or the second hollow portion 2026 provide a region that provides knotless fixation via engagement between one or more components of the suture construct 2010, as described herein.

For example, in some embodiments the first hollow portion 2024 may be a first finger trap region and/or the second hollow portion 2026 may be a second finger trap region, for example to one or provide finger trap regions for fixation (e.g., via a structural characteristic or configuration of the region, upon deployment of the suture construct 2010, etc.), as described elsewhere herein. For example, the working suture 2020 (e.g., the sliding tail 2022) may be shuttled through the first hollow portion 2024 and/or a force (e.g., tension) may be applied to the working suture 2020 (e.g., the sliding tail 2022, etc.), such that the first hollow portion 2024 provides a knotless fixation region. In some embodiments, the first hollow portion 2024 provides a knotless fixation region via a positioning of the working suture 2020 (e.g., the sliding tail 2022, etc.) within the first hollow portion 2024. In other embodiments, the first hollow portion 2024 provides a knotless fixation region via an engagement (e.g., high-friction engagement, a co-axial engagement of suture material, etc.) between suture material at the first hollow portion 2024 (e.g., the working suture 2020 within the first hollow portion 2024, etc.). It should be understood that it is contemplated that in other embodiments, the first hollow portion 2024 and/or the second hollow portion 2026 provide other forms of knotless fixation regions and/or other knotless fixation, as described elsewhere herein. All such embodiments are contemplated herein.

In an example embodiment, the first hollow portion 2024 and/or the second hollow portion 2026 are defined by one or more openings, slits, and/or splices (e.g., an entry opening, an exit opening, etc.), for example to facilitate passage and/or movement of one or more sutures, constructs, and/or components (e.g., through the first hollow portion 2024, the second hollow portion 2026, etc.), as described herein. For example, and as shown in at least FIG. 27B, the first hollow portion 2024 is shown to include or be defined by an entry opening 2028 and an exit opening 2029, and the second hollow portion 2026 is shown to include or be defined by at least an opening 2027.

In an exemplary embodiment, in a first configuration or a pre-deployment configuration, one or more components of the working suture 2020 are positioned at and/or along portions of the working suture 2020. For example, in a pre-deployment configuration a distance between the end of the fixed tail 2021 (e.g., the fixed tip 2011, etc.) and the first hollow portion 2024 (e.g., the entry opening 2028) may be approximately 26 inches; however, in other embodiments the distance is another suitable distance (e.g., approximately 20, 25, 30, 35, etc. inches). Further, in a pre-deployment configuration the first hollow portion 2024 may define a distance (e.g., between the entry opening 2028 and the exit opening 2029, etc.) of approximately 23 millimeters; however, in other embodiments a distance of the first hollow portion 2024 is another suitable distance (e.g., approximately 15, 20, 25, 30, etc. millimeters). According to an exemplary embodiment, and as described herein, upon deployment the first hollow portion 2024 (and/or one or more sutures or components contained therein, etc.) may provide a knotless fixation region, such as a finger trap region, a finger trap, and/or another suitable region comprising one or more knotless fixation components or configurations.

Referring to FIGS. 27A-27B, the tucked suture 2030 is shown to include a loop or ring, shown as a tucked loop 2032, and an end, tip, or tail, or a tucked tail 2034 (e.g., shown in FIGS. 27A-27B as being within the working suture 2020, etc.). As shown, the tucked loop 2032 is external to the working suture 2020, and may be formed via a Brummel splice, and/or any other suitable technique for providing a fixed eyelet. In some embodiments, the tucked loop 2032 is formed via a sliding eyelet, such as a Munter hitch, a sliding eyelet that closes during tensioning, and/or another suitable technique for providing a sliding eyelet, as described herein. In some embodiments, the tucked loop 2032 is prepared with an opening that is between 0.5 and 3.0 millimeters in diameter; however, in other embodiments the tucked loop 2032 is prepared with another suitable sized and/or shaped opening (e.g., approximately 1, 2, 2.5, 3.5, 5, etc. millimeters in diameter, etc.). In some embodiments, the tucked suture 2030 is prepared such that the tucked loop 2032 is partially internal relative to the working suture 2020 (e.g., the second hollow portion 2026, etc.), for example in a pre-deployment configuration. In other embodiments, the tucked loop 2031 may be completely internal with respect to the working suture 2020.

In an exemplary embodiment, the tucked tail 2034 (e.g., extending from the tucked loop 2032) passes into, though, and/or within the second hollow portion 2026 of the working suture 2020 (e.g., coaxially, coaxially within the second hollow portion 2026, etc.), for example via the opening 2027. The tucked tail 2034 may be secured relative to the working suture 2020 (e.g., within the second hollow portion 2026, etc.), for example via an eyelet or splice, shown as splice 2035, positioned along at least one location of its length. In some embodiments, the tucked tail 2034 is secured within the second hollow portion 2026 (e.g., via the splice 2035), while also extending within the second hollow portion 2026 beyond the splice 2035 (e.g., as shown in at least FIG. 27B). As shown, the tucked tail 2034 may extend within the working suture 2020, such that the tucked tail 2034 defines a length, which may be approximately 3.5 inches and/or another suitable length (e.g., approximately 2.5, 3, 4, 5, 7, etc. inches). In an example embodiment, the length of the tucked tail 2034 is such that when the sliding tail 2022 is passed to and/or through the first hollow portion 2024, a portion of the tucked tail 2034 (e.g., within the second hollow portion 2026, the sliding tail 2022, etc.) also passes through the first hollow portion 2024 (e.g., and the tucked loop 2032, etc.), as also described elsewhere herein. In an example embodiment, the tucked suture 2030 is a 2-0 size suture; however, in other embodiments the tucked suture 2030 is another suitable size (e.g., 3-0, 0, 1, etc. size), for example to allow the sliding tail 2022 (e.g., and the tucked suture 2030 portion of the sliding tail 2022, etc.) to pass through the first hollow portion 2024 (e.g., coaxially through the first hollow portion 2024, etc.) and/or the tucked loop 2032.

Referring still to FIG. 27A, the shuttle 2060 is shown to include a first end, portion, or loop, shown as a shuttle loop 2062, and a second end, portion, or tail, shown as shuttle tail 2066. As described herein, the shuttle 2060 may be formed of suture (e.g., a size 0 suture, a size 3-0 suture, another suitable size suture, including 2-0, 0, 1, size, etc.), nitinol wire, suture with monofilament, and/or any suitable combination thereof. For example, and as will be described in greater detail herein, the shuttle loop 2062 may be primarily formed of a size 3-0 suture (e.g., with a size 0 suture loop portion, etc.), while a body of the shuttle 2060, including shuttle tail 2066, may be formed of a size 0 suture.

As shown, the shuttle loop 2062 includes a tucked portion, shown as tucked shuttle loop 2063. The tucked shuttle loop 2063 may be formed by passing the shuttle loop 2062 into, through, and/or within the working suture 2020 (e.g., via an opening or splice, etc.). In an example embodiment, the tucked shuttle loop 2063 is formed by passing the shuttle loop 2062 into the working suture 2020 a distance or length from an end of the working suture 2020. For example, in a pre-deployment configuration a distance between the end of the fixed tail 2021 (e.g., the fixed tip 2011, etc.) and the tucked shuttle loop 2063 and/or the shuttle loop 2062 may be approximately 7.5 inches; however, in other embodiments the distance may be another suitable distance (e.g., e.g., 2.5, 5, 10, 12.5, etc. inches).

As shown, the shuttle 2060 may extend from the shuttle loop 2062 and to the first hollow portion 2024, for example external to the working suture 2020 (e.g., as shown in at least FIG. 27A, etc.). In an exemplary embodiment, the shuttle 2060 may extend into and/or within the first hollow portion 2024 (e.g., via the entry opening 2028, etc.), out the first hollow portion 2024 (e.g., via the exit opening 2029, etc.), and/or through the tucked loop 2032, for example leaving at least a portion of the shuttle 2060 and/or the shuttle tail 2066 external to the working suture 2020 (e.g., as shown in at least FIGS. 27A-B). As discussed herein, the first hollow portion 2024 may provide a knotless fixation region (e.g., a finger trap region, a finger trap upon deployment, etc.), where a length of the knotless fixation region may factor into the overall securement strength of the suture construct 2010 (e.g., with a longer length being generally associated with a higher securement force, etc.). As shown, the shuttle tail 2066 may extend from the first hollow portion 2024 (e.g., via the exit opening 2029, etc.) a distance. In an example embodiment, the shuttle tail 2066 is approximately 4 inches; however, in other embodiments the shuttle tail 2066 has another suitable length (e.g., 2, 3, 5, 6, 10, etc. inches). In some embodiments, the shuttle tail 2066 includes a grasp, hold, or grip, shown as knot 2067 for example, along a length of the shuttle tail 2066. The knot 2067 may be positioned at a middle of the shuttle tail 2066, for example to facilitate manipulating (e.g., grasping, holding, etc.) the shuttle tail 2066 during deployment of the suture construct 2010.

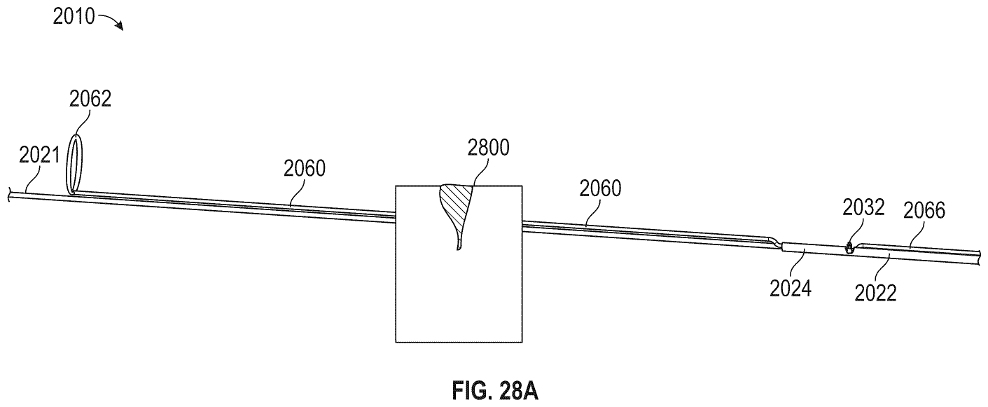

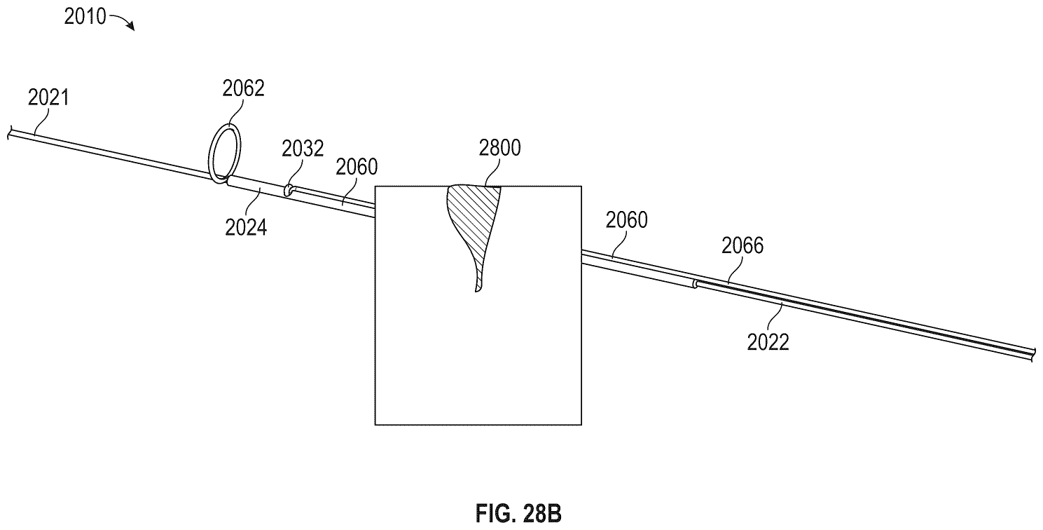

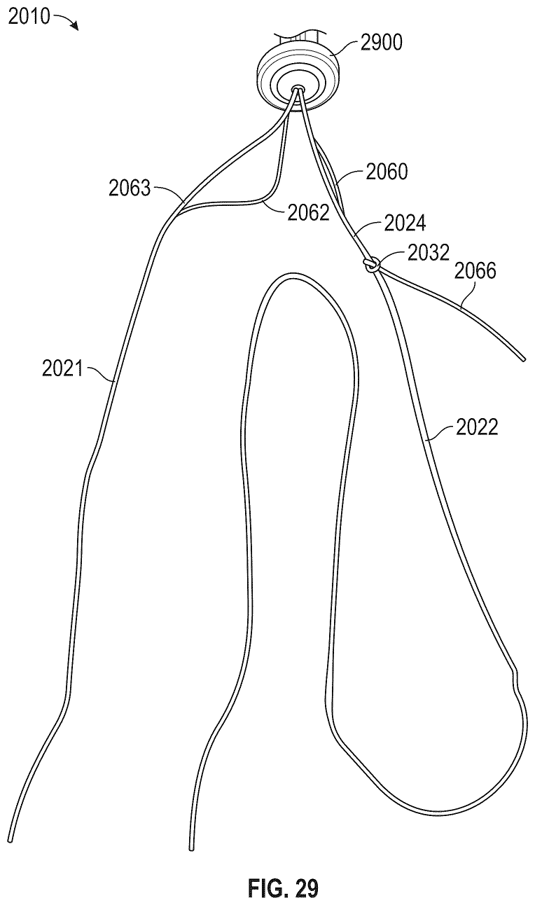

Referring generally to FIGS. 28A-32, the suture construct 2010 may be deployable, for example for the closure of a tissue tear and/or other gap (e.g., in the tissue, etc.), as described herein. In an example embodiment, the suture construct 2010 is deployable between a series of configurations, for example to manipulate the suture construct 2010 between a first or pre-deployment configuration (e.g., as shown in at least FIG. 27A) and a second or deployed configuration (e.g., as shown in at least FIGS. 29-32).

Referring now to FIG. 28A, the suture construct 2010 is provided, for example to repair a tissue, shown as tissue 2800. In an example embodiment, the tissue 2800 includes a tear, such that the tissue 2800 includes opposing capsules, flaps, or portions of tissue. As shown, an end of the suture construct 2010 may be inserted into and/or through the tissue 2800 (e.g., through opposing tissue capsules, flaps, or portions, etc.). For example, the fixed tail 2021 may be inserted into and/or through the tissue 2800. Similarly, in other embodiments, the sliding tail 2022 is inserted into and/or through the tissue 2800. In some embodiments, an end of the suture construct 2010 is inserted into and/or through the tissue 2800 arthroscopically with use of a cannula. According to an exemplary embodiment, and as shown in FIG. 28A, with one end of the suture construct 2010 inserted into and through the tissue 2800, the ends of the suture construct 2010 (e.g., the fixed tail 2021, the sliding tail 2022, a combination thereof, etc.) may be manipulated such that the suture construct 2010 is positioned with the shuttle loop 2062 and the shuttle tail 2066 on opposing sides of the tissue 2800 (e.g., opposite sides of a tear or gap in the tissue 2800, etc.).

As shown in FIG. 28A, the suture construct 2010 is also positioned with the shuttle loop 2062 and the first hollow portion 2024 on opposing sides of the tissue 2800 (e.g., opposite sides of a tear or gap in the tissue 2800, etc.). Advantageously, the suture construct 2010 and/or configuration shown in at least FIG. 28A allows for deployment of the suture construct 2010 with less force (e.g., shuttling force, tension, etc.) compared to other suture construct configurations (e.g., where the shuttle loop 2062, the first hollow portion 2024, and/or the knotless fixation region or finger trap region are on the same side of the tissue 2800, etc.). For example, and as described herein, during deployment the sliding tail 2022 is shuttled through the tissue 2800 (e.g., via the shuttle loop 2062 and/or the shuttle 2060, etc.) and then through the first hollow portion 2024 (e.g., where the first hollow portion 2024 provides a knotless fixation region, such as a finger trap portion, etc.), which results in less force needed to shuttle the sliding tail 2022 (e.g., through the tissue 2800 and then through the first hollow portion 2024, compared to shuttling the sliding tail 2022 and the first hollow portion 2024, through the tissue 2800, etc.).

Further, by positioning the shuttle loop 2062 and the first hollow portion 2024 on opposing sides of the tissue 2800, the suture construct 2010 and/or configuration shown in at least FIG. 28A allows for more control over the positioning and/or setting of the first hollow portion 2024 (e.g., relative to the tissue 2800, for example via manipulation of the fixed tail 2021 to pull the opening or entrance of the first hollow portion 2024 back toward the tissue 2800, for example without providing a knotless fixation engagement or finger trap at the first hollow portion 2024, etc.), and/or less tangling at the first hollow portion 2024 (e.g., the sliding tail 2022 and the first hollow portion 2024 are pulled away from the tissue 2800 during shuttling, compared to toward and/or through the tissue 2800 during shuttling which in some instances may result in tangling or snagging during shuttling, etc.). Yet further, the suture construct 2010 and/or the configuration shown in at least FIG. 28A may also allow for shuttling the sliding tail 2022 through the first hollow portion 2024 to occur outside a cannula and/or away from the tissue 2800, which results in less force needed for shuttling, improved control over positioning of the first hollow portion 2024, and/or reduced tangling as a result of shuttling or deployment.

Referring now to FIG. 28B, the suture construct 2010 is provided in an alternative configuration, for example to repair a tissue (e.g., the tissue 2800). In an example embodiment, and as described herein, the tissue 2800 may include a tear, such that the tissue 2800 includes opposing capsules, flaps, or portions of tissue. As shown, an end of the suture construct 2010 may be inserted into and/or through the tissue 2800 (e.g., through opposing tissue capsules, flaps, or portions, etc.). For example, the fixed tail 2021 may be inserted into and/or through the tissue 2800. Similarly, in other embodiments, the sliding tail 2022 is inserted into and/or through the tissue 2800.

According to an exemplary embodiment, and as shown in FIG. 28B, with one end of the suture construct 2010 inserted into and through the tissue 2800, the ends of the suture construct 2010 (e.g., the fixed tail 2021, the sliding tail 2022, a combination thereof, etc.) may be manipulated such that the suture construct 2010 is positioned with the shuttle loop 2062 and the shuttle tail 2066 on the same side of the tissue 2800 (e.g., the same side of a tear or gap in the tissue 2800, etc.). As shown in FIG. 28B, the suture construct 2010 is also positioned with the shuttle loop 2062 and the first hollow portion 2024 on the same side of the tissue 2800 (e.g., the same side of a tear or gap in the tissue 2800, etc.).

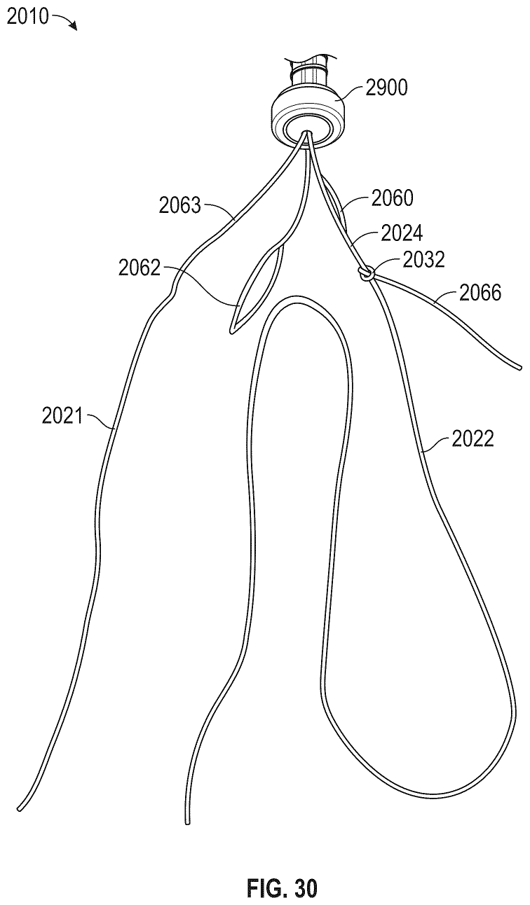

Referring now to FIGS. 29-32, with the suture construct 2010 positioned relative to the tissue 2800 (e.g., as shown in at least FIG. 28), the suture construct 2010 may be deployed in one or more configurations. FIGS. 29-32 illustrate one example of how the suture construct 2010 is available and manipulatable by an operator outside of a surgical cannula, shown as a cannula 2900. It should be understood that while the tissue 2800 is not shown, it should be appreciated that the tissue 2800 may be positioned adjacent to or near the other end of the cannula 2900. For example, in at least one exemplary configuration, the operator might separate the tucked shuttle loop 2063 and/or the working suture 2020 (e.g., as shown in at least FIG. 29) from one another, such that shuttle loop 2062 can be utilized as described below. In an exemplary embodiment, the shuttle loop 2062 is split from the working suture 2020, for example by manipulating (e.g., pulling, forcing, etc.) a portion of the tucked shuttle loop 2063 out of the working suture 2020. In this sense, the shuttle loop 2062 may be accessible for use by pulling at least a portion of the tucked shuttle loop 2063 out of the working suture 2020 (e.g., as shown in at least FIG. 30), thereby separating the shuttle loop 2062 from working suture 2020.

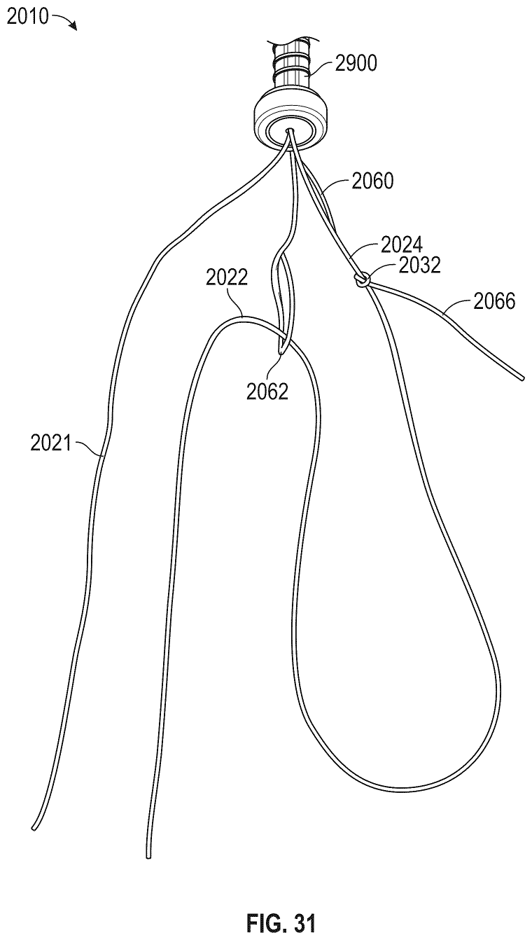

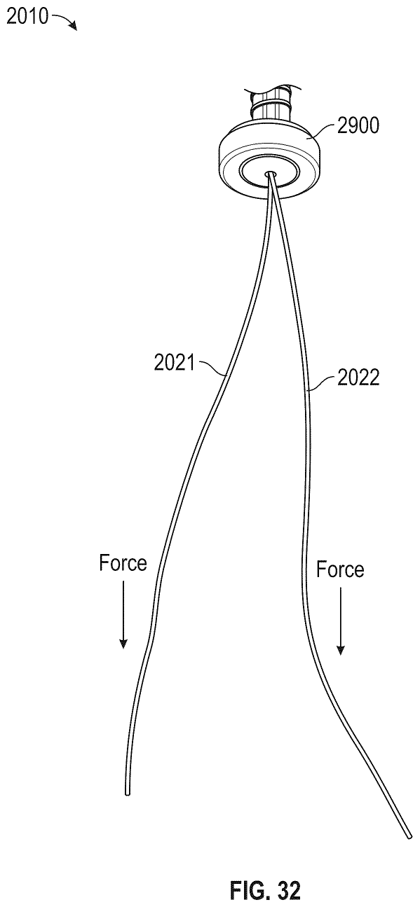

With the shuttle loop 2062 split or separated from the working suture, the sliding tail 2022 may engage the shuttle loop 2062. In an exemplary embodiment, the sliding tail 2022 is looped through the shuttle loop 2062 (e.g., as shown in at least FIG. 31). The shuttle tail 2066 may then be manipulated (e.g., pulled, etc.), thereby moving the shuttle loop 2062 (and the sliding tail 2022) through the tissue 2800 and toward the first hollow portion 2024. As discussed elsewhere herein, the manipulation of the shuttle tail 2066 further moves the shuttle loop 2062 (and the sliding tail 2022) into and through the first hollow portion 2024 of the working suture 2020, through the tucked loop 2032 of the tucked suture 2030, and/or exterior to the working suture 2020. With the sliding tail 2022 shuttled through the working suture 2020 (e.g., the first hollow portion 2024, etc.), the sliding tail 2022 and/or the working suture 2020 can be manipulated (e.g., pulled, etc., for example as shown in at least FIG. 32, etc.), for example to form a loop about the tissue 2800 (e.g., the opposing tissue capsules, flaps, portions, etc.) to repair the tissue 2800.

In an exemplary embodiment, and as described elsewhere herein, the shuttle tail 2066 (and/or the sliding tail 2022) and/or the working suture 2020 may be manipulated (e.g., pulled, etc.) with sufficient force, such that a knotless fixation region or finger trap is provided within and/or at the first hollow portion 2024 (e.g., due to the nature of the first hollow portion 2024, and/or due to the sliding tail 2022 and/or the tucked suture 2030 portion of the sliding tail 2022 being positioned within the first hollow portion 2024 of the working suture 2020, etc.), and/or the tucked loop 2032 may couple (e.g., cinch, etc.) the working suture 2020. FIG. 32 illustrates the result of the method of FIGS. 29-31, whereby tails 2021, 2022 remain outside of the patient and the operator may tension one or both tails to tighten the fixation of working suture 2020. As described elsewhere herein, the knotless fixation region may be a finger trap, for example provided via the nature of the first hollow portion 2024 and/or via a one-way tensioning mechanism that is characterized by a high-friction engagement created between co-axial suture material.

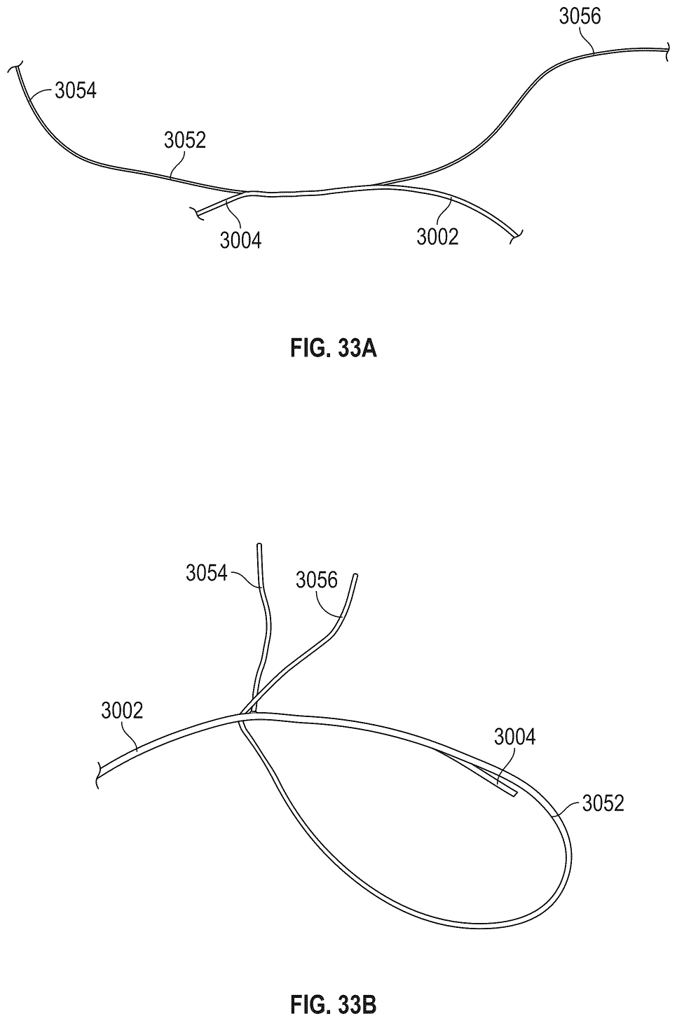

Referring now to FIGS. 33A-37, components of the suture construct 2010 are shown in greater detail, according to example embodiments. In an example embodiment, FIGS. 33A-36 illustrate example methods or processes of forming the shuttle 2060. As described herein, the shuttle 2060 may include the shuttle loop 2062, which may be formed of a size 3-0 suture and/or a size 0 suture, while a body of the shuttle 2060 may be formed of a size 0 suture. Advantageously, the size of the body of the shuttle 2060 (e.g., size 0 suture, etc.) may allow for easy handling and/or manipulation (e.g., by a surgeon or operator, etc.), while the size of at least a portion of the shuttle loop 2062 (e.g., size 3-0 suture, which is narrower than the size 0 suture, etc.) allows for easier shuttling (e.g., through the tissue 2800, the working suture 2020, etc.), as described herein. It should be understood that it is contemplated that various embodiments the shuttle 2060 and/or the components thereof (e.g., a body, the shuttle loop 2062, etc.) may be or include any other suitable size sutures, and/or combinations thereof (e.g., 2-0, 1, etc. size sutures).

Referring generally to FIGS. 33A-33B, a first example method or process of forming the shuttle 2060 is shown, according to an example embodiment. As shown, the shuttle 2060 may be formed using a first suture portion or piece, shown as first suture 3002, and a second suture portion or piece, shown as second suture 3052. The first suture 3002 may form a body of the shuttle 2060, and/or may be a size 0 suture. The second suture 3052 may be used to form at least a portion of the shuttle loop 2062 (e.g., a portion of a loop, etc.), and/or may be a size 3-0 suture. As shown in at least FIG. 33A, the second suture 3052 may be passed into, through, and/or within the first suture 3002 (e.g., coaxially within the first suture 3002, etc.). For example, and as shown in at least FIG. 33A, the second suture 3052 may be passed into, through, and/or within the first suture 3002 (e.g., via one or more openings and/or exits, etc.), such that the second suture 3052 has a first end or portion, shown as first tail 3054, and a second end or portion, shown as second tail 3056, extending from and/or out of opposing portions of the first suture 3002. The second suture 3052 is also shown to pass into, through, and/or within a central or middle portion of the first suture 3002 (e.g., a portion offset or away from an end of the first suture 3002, etc.), such that the first suture 3002 has a first end or portion, shown as first tail 3004 (e.g., a short tail, etc.).

According to an exemplary embodiment, the shuttle 2060 may be formed by providing one or more holes, openings, apertures, or piercings within the second suture 3052. For example, with the second suture 3052 passed through the first suture 3002, a first opening or piercing may be provided in the first tail 3054, and a second opening or piercing may be provided in the second tail 3056. The first opening of the first tail 3054 may be offset from an end of the first tail 3054 and the second opening of the second tail 3056 may be offset from an end of the second tail 3056. As shown in at least FIG. 33B, the first suture 3002 may be passed into and/or through the first opening of the first tail 3054 and the second opening of the second tail 3056. For example, the free end or end opposite the first tail 3004 of the first suture 3002 may be manipulated such that the first suture 3002 passes into and/or through the first opening of the first tail 3054 and the second opening of the second tail 3056 (e.g., as shown in at least FIG. 33B).

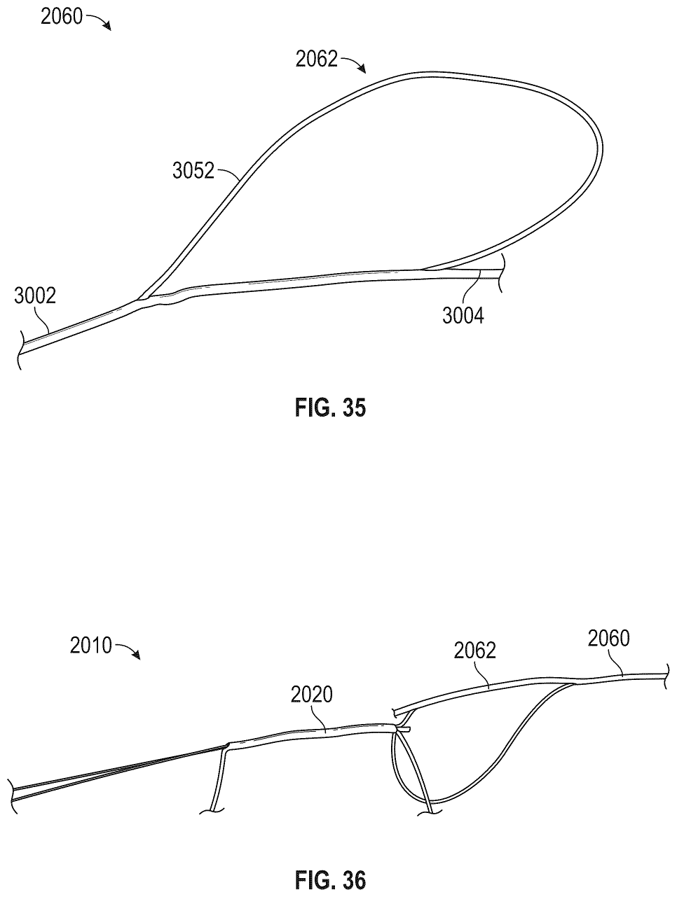

With the first suture 3002 passed through the openings of the first tail 3054 and the second tail 3056, the ends of the first tail 3054 and the second tail 3056 may be secured to form the shuttle 2060 having the shuttle loop 2062 (e.g., as shown in at least FIG. 35). For example, the ends of the first tail 3054 and the second tail 3056 may be passed back into, through, and/or within the first suture 3002, thereby forming the shuttle loop 2062. In some embodiments, the ends of the first tail 3054 and the second tail 3056 are coupled and/or trimmed, for example to form the shuttle loop 2062. In yet other embodiments, the ends of the first tail 3054 and the second tail 3056 are otherwise coupled and/or modified relative to the first suture 3002, for example to form the shuttle 2060 described herein.

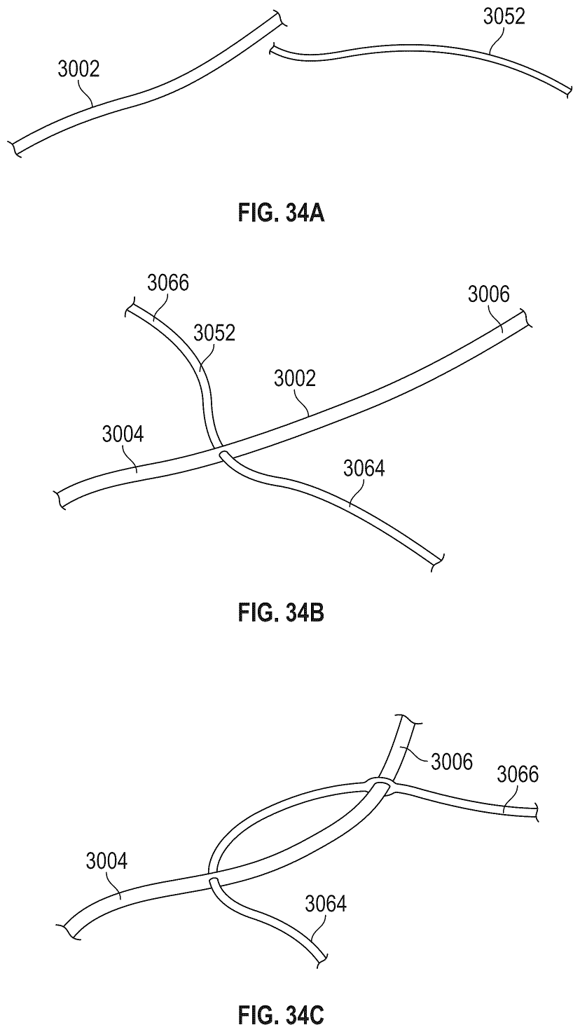

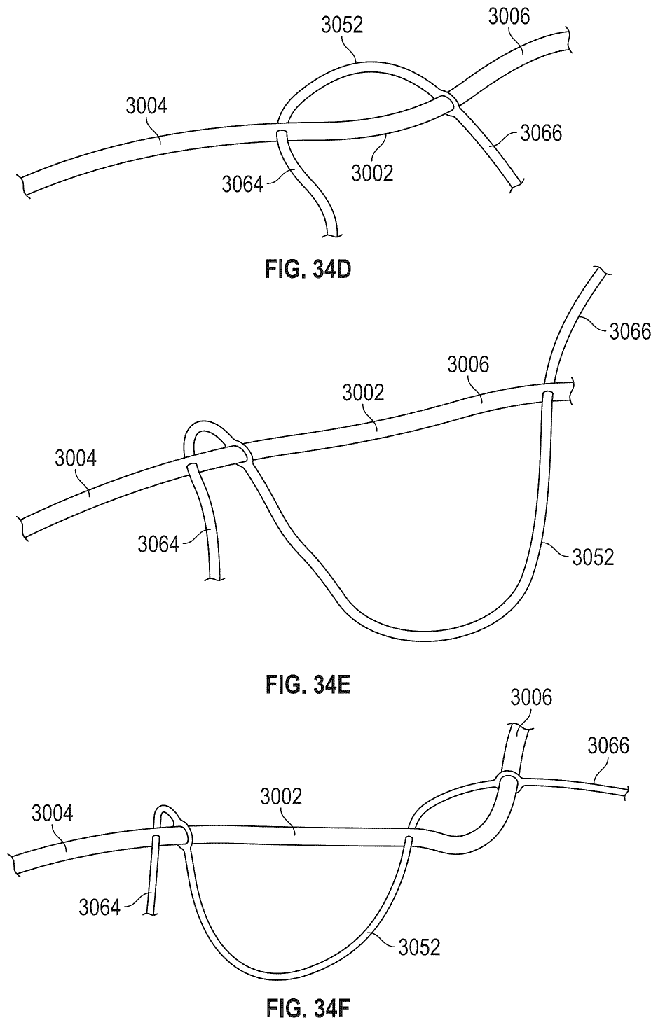

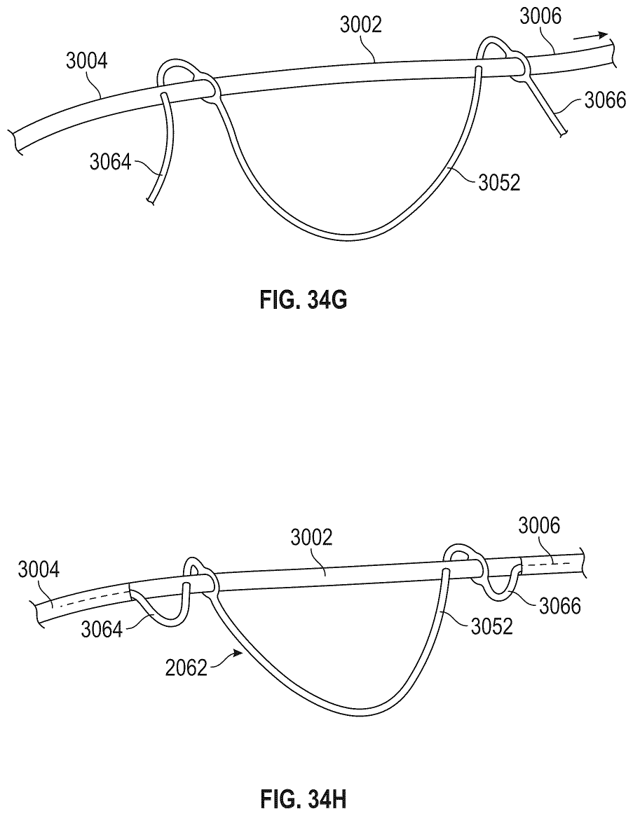

Referring now generally to FIGS. 34A-34H, a second example method or process of forming the shuttle 2060 is shown, according to an example embodiment. As shown in at least FIG. 34A, the shuttle 2060 may be formed using a first suture portion or piece, shown as first suture 3002, and a second suture portion or piece, shown as second suture 3052. As discussed herein, the first suture 3002 may constitute a body of the shuttle 2060, and/or may be any size desired, such as a size 0 suture. The second suture 3052 may establish at least a portion of the shuttle loop 2062 (e.g., a portion of a loop, etc.), and/or may be any size desired, such as a size 3-0 suture.

As shown in at least FIG. 34B, the shuttle 2060 may be formed by providing one or more holes, openings, apertures, or piercings within the first suture 3002. For example, a first opening or piercing may be provided in the first suture 3002. Typically, where the suture is a braided suture formed of a plurality of braided filaments, the opening or piercing is formed by establishing a pathway through the suture body and in between the strands. For example, the shuttle 2060 may be passed through the body of first suture 3002 using a needle or other instrument to establish a pathway through the various strands forming braided suture 3002 to establish shuttle 2060 passing through an opening or piercing in suture 3002. As shown in at least FIG. 34B, the second suture 3052 may be passed into and/or through the first opening of the first suture 3002, such that the second suture 3052 has a first end or portion, shown as first tail 3064, and a second end or portion, shown as second tail 3066, extending from and/or out of opposing portions of the first suture 3002. Further, the second suture 3052 may be passed through the first suture 3002, such that the first suture has a first end or portion, shown as the first tail 3004 (e.g., a short tail, etc.) and a second tail 3006 (e.g., a long tail, etc.).

Further, and as shown in at least FIG. 34C, the shuttle 2060 may be formed by providing one or more holes, openings, apertures, or piercings within the second suture 3052 (which may be formed in a similar manner as discussed above). For example, a first opening or piercing may be provided in the second tail 3066 of the second suture 3052. As shown in at least FIG. 34C, the first suture 3002 may be passed into and/or through the first opening in the second suture 3052. For example, the second tail 3006 of the first suture 3002 may be passed through the first opening of the second tail 3066 of the second suture 3052.

According to an exemplary embodiment, with the second suture 3052 passed through the first suture 3002 (e.g., as shown in at least FIG. 34B) and the first suture 3002 passed through the second suture 3052 (e.g., as shown in at least FIG. 34C), one or more of the sutures may be manipulated to position the splices or openings (e.g., as shown in at least FIG. 34). For example, in an exemplary embodiment a force may be applied (e.g., tension, pulling, etc.) to the first tail 3064 of the second suture 3052 to position (e.g., move, etc.) the splices toward one another. In some embodiments, the second tail 3006 of the first suture 3002 may be manipulated, for example to position (e.g., move, etc.) the splices toward one another.

As shown in at least FIG. 34E, with the splices or openings positioned adjacent one another, the shuttle 2060 may be formed by providing one or more additional holes, openings, apertures, or piercings within the first suture 3002. For example, a second opening or piercing may be provided in the first suture 3002. According to an exemplary embodiment, a second opening or piercing is provided in the second tail 3006 of the first suture 3002. For example, a second opening or piercing may be provided in the second tail 3006 approximately 1 inch from the first opening or piercing in the second tail 3006, In other embodiments, the second opening or piercing is provided another suitable distance from the first opening or piercing (e.g., approximately 0.5, 1.5, 2, 3, 5, 7.5, etc. inches). As shown in at least FIG. 34E, the second suture 3052 (e.g., the second tail 3066, etc.) may be passed into and/or through the second opening of the first suture 3002 (e.g., the second tail 3006).

Further, and as shown in at least FIG. 34F, the shuttle 2060 may be formed by providing one or more additional holes, openings, apertures, or piercings within the second suture 3052. For example, a second opening or piercing may be provided in the second tail 3066 of the second suture 3052 (e.g., a distance from the first opening or piercing in the second tail 3066, etc.). As shown in at least FIG. 34F, the first suture 3002 may be passed into and/or through the second opening in the second suture 3052. For example, the second tail 3006 of the first suture 3002 may be passed through the second opening of the second tail 3066 of the second suture 3052.

In this regard, and as illustrated in at least FIGS. 34B-34F, the shuttle 2060 may be formed by passing the second suture 3052 through a first opening in the first suture 3002 (e.g., as shown in at least FIG. 34B), passing the first suture 3002 through a first opening in the second suture 3052 (e.g., as shown in at least FIG. 34C), manipulating one or more of the sutures to position the openings and/or splices adjacent one another (e.g., as shown in at least FIGS. 34D-34E), passing the second suture 3052 through a second opening in the first suture 3002 a distance away from the first opening(s) (e.g., as shown in at least FIG. 34E), and then passing the first suture 3002 through a second opening in the second suture 3052 (e.g., as shown in at least FIG. 34F).

As shown in at least FIG. 34G, in an exemplary embodiment one or more of the sutures may be manipulated to position the second splices or openings. For example, in an exemplary embodiment a force may be applied (e.g., tension, pulling, etc.) to the second tail 3006 of the first suture 3002 to position (e.g., move, etc.) the second splices toward one another. In some embodiments, the second tail 3066 of the second suture 3052 and/or the second suture 3052 itself may be manipulated, for example to position (e.g., move, etc.) the second splices toward one another. In such a configuration, the first suture 3002 and the second suture 3052 may form a loop (e.g., between the first openings or splices and the second openings or splices, etc.), for example of the shuttle loop 2062.

According to an exemplary embodiment, the ends of the first tail 3064 and the second tail 3066 may be secured to form the shuttle 2060 having the shuttle loop 2062 (e.g., as shown in at least FIG. 34H). For example, the ends of the first tail 3064 and the second tail 3066 may be passed back into, through, and/or within the first suture 3002 (e.g., the first tail 3004 and the second tail 3006, respectively), thereby forming the shuttle loop 2062 (e.g., as shown in at least FIG. 34H). According to various embodiments, one or both of the first tail 3004 and/or the second tail 3006 may be passed back into, through, or within the first suture 3002, for example in a direction opposite to that shown in FIG. 34H. In some embodiments, the ends of the first tail 3064 and the second tail 3066 are coupled and/or trimmed, for example to form the shuttle loop 2062. In yet other embodiments, the ends of the first tail 3064 and the second tail 3066 are otherwise coupled and/or modified relative to the first suture 3002, for example to form the shuttle 2060 described herein.

Advantageously, the size of the body of the shuttle 2060 (e.g., size 0 suture of the first suture 3002, etc.) may allow for easy handling and/or manipulation (e.g., by a surgeon or operator, etc.), while the size of the shuttle loop 2062 (e.g., size 3-0 suture of the second suture 3052, etc.) allows for easier shuttling (e.g., through the tissue, the working suture 2020, etc.). Further, and as shown in FIG. 35, the shuttle 2060 may be formed such that the shuttle loop 2062 is an asymmetric loop or ring, for example to allow for easy handling and/or manipulation by a surgeon or operator (e.g., without requiring opening or splicing the shuttle loop 2062 by the operator during deployment or operation, etc.). For example, the shuttle 2060 may be formed such that the shuttle loop 2062 remains open (e.g., automatically, without operator intervention, etc.) to allow for easy handling and/or manipulation by a surgeon or operator.

Further, and as described herein, in some embodiments the shuttle 2060 includes the first tail 3004. As shown in at least FIG. 35, the first tail 3004 is shown to extend a distance from a portion of the shuttle loop 2062 (e.g., an interface or junction between the first suture 3002 and the second suture 3052). According to an exemplary embodiment, the first tail 3004 extends a distance that is shorter than an approximate length of the shuttle loop 2062 when the shuttle loop 2062 is in a flattened or tucked configuration (e.g., flattened in line with the first suture 3002, tucked within the working suture 2020, tucked within the working suture 2020 to provide the tucked shuttle loop 2063, etc.). In an exemplary embodiment, the first tail 3004 extends a distance that is shorter than an approximate length of the shuttle loop 2062 when the shuttle loop 2062 is in a flatted or tucked configuration, such that when the shuttle loop 2062 receives the sliding tail 2022 (e.g., in the tucked or flatted configuration, when tucked within the working suture 2020, etc.) cross-sectional thicknesses of the first tail 3004 and the shuttle loop 2062 (e.g., including the sliding tail 2022, etc.) are approximately the same. In such a configuration, the first tail 3004 and/or the shuttle loop 2062 allow for easier shuttling (e.g., through the tissue, through the working suture 2020, the first hollow portion 2024, the tucked loop 2032, etc.) of the shuttle 2060, the shuttle loop 2062, and/or the sliding tail 2022 (e.g., due to similar cross-sectional thicknesses, etc.).

some embodiments, the first tail 3004 extends a distance that is greater than a length of the shuttle loop 2062 when the shuttle loop 2062 is in a flatted or tucked configuration (e.g., flattened in line with the first suture 3002, tucked within the working suture 2020, tucked within the working suture 2020 to provide the tucked shuttle loop 2063, etc.). In such a configuration, the first tail 3004 may extend from the working suture 2020 and/or provide a point for manipulation by a user or operator (e.g., grasping, grabbing, manipulating, etc.), for example to more easily expose a portion of the tucked shuttle loop 2063 and/or the shuttle loop 2062 (e.g., from within the working suture 2020, etc.) to receive the sliding tail 2022. Yet further, such a configuration may allow for easier formation of the shuttle loop 2062 using the techniques described herein, for example by allowing for easier positioning and/or manipulation of the first suture 3002 (and/or the associated first tail 3004, etc.).

In other embodiments, the first tail 3004 extends a distance that is approximately the same length of the shuttle loop 2062 when the shuttle loop 2062 is in a flattened or tucked configuration. In yet other embodiments, the first tail 3004 extends a distance that is somewhere between approximately 0 inches and the length of the shuttle loop 2062 when the shuttle loop 2062 is in the flatted or tucked configuration. It should be understood that all such embodiments are contemplated herein, for example to allow for easier shuttling (e.g., of the shuttle 2060, the shuttle loop 2062, and/or the sliding tail 2022, etc.), easier manipulation and/or exposure of the shuttle loop 2062, and/or easier manufacturing of the shuttle 2060 and/or the components thereof (e.g., the shuttle loop 2062, etc.). It should also be understood that it is contemplated that in some embodiments, the shuttle 2060 does not include the first tail 3004 (e.g., the first tail 3004 has a length of approximately 0 inches, etc.).

In some embodiments, the shuttle 2060 and/or the components thereof (e.g., the shuttle loop 2062, etc.) may be formed using other suitable configurations and/or techniques.

As an example, and as described herein, in some embodiments the shuttle 2060 includes a first tail (e.g., the first tail 3004, etc.) extending from or beyond the shuttle loop 2062. In some embodiments, the shuttle loop 2062 may be formed by passing a first end of the second suture 3052 (e.g., the first tail 3054) into and through the first suture 3002 (e.g., coaxially, etc.) in a first direction, and a second end of the second suture 3052 (e.g., the second tail 3056) into and through the first suture 3002 (e.g., coaxially) a distance from the first end and in a second direction (e.g., opposite the first direction, etc.). In such embodiments, portions of the second suture 3052 (e.g., the first tail 3054, the second tail 3056, etc.) overlap within a portion of the first suture 3002, and the second suture 3052 forms the shuttle loop 2062 external to the first suture 3002 while a portion of the first suture 3002 forms a tail (e.g., the first tail 3004). In other embodiments, the shuttle loop 2062 may be formed by passing a first end of the second suture 3052 (e.g., the first tail 3054) into the first suture 3002 (e.g., coaxially, etc.) in a first direction and out an end of the first suture 3002, and a second end of the second suture 3052 (e.g., the second tail 3056) into and through the first suture 3002 (e.g., coaxially) a distance from the first end and in a second direction (e.g., opposite the first direction, etc.). In such embodiments, portions of the second suture 3052 (e.g., the first tail 3054, the second tail 3056, etc.) overlap within a portion of the first suture 3002, and the second suture 3052 forms the shuttle loop 2062 external to the first suture 3002 while a portion of the second suture 3052 forms a tail (e.g., the end of the first tail 3054 that extends from the end of the first suture 3002, etc.). All such embodiments and techniques are contemplated herein, which, for example, provide the advantages described elsewhere herein.

As another example, and as also described herein, in some embodiments the shuttle 2060 does not include a first tail (e.g., the first tail 3004, etc.). In some embodiments, the shuttle loop 2062 may be formed by passing a first end of the second suture 3052 (e.g., the first tail 3054) into and through the first suture 3002 (e.g., coaxially, etc.) in a first direction, and a second end of the second suture 3052 (e.g., the second tail 3056) into and through an end of the first suture 3002 (e.g., coaxially) in a second direction (e.g., opposite the first direction, etc.), such that portions of the second suture 3052 (e.g., the first tail 3054, the second tail 3056, etc.) overlap within a portion of the first suture 3002, and the second suture 3052 forms the shuttle loop 2062 external to the first suture 3002 (e.g., extending from the end of the first suture 3002, etc.). In other embodiments, the shuttle loop 2062 may be formed by passing a first end of the second suture 3052 (e.g., the first tail 3054) into and through the first suture 3002 (e.g., coaxially, etc.) in a first direction, and a second end of the second suture 3052 (e.g., the second tail 3056) into and through and end of the first suture 3002 (e.g., coaxially, etc.) in the first direction, such that portions of the second suture 3052 (e.g., the first tail 3054, the second tail 3056, etc.) extend within a portion of the first suture 3002 in the same direction, and the second suture 3052 forms the shuttle loop 2062 external to the first suture 3002 (e.g., extending from the end of the first suture 3002). In such embodiments, the end of the first suture 3002 has a tapered configuration (e.g., a cross-sectional thickness that decreases as you move along the first suture 3002, etc.), for example to allow for easier shuttling (e.g., through the tissue, through the working suture 2020, the first hollow portion 2024, the tucked loop 2032, etc.) of the shuttle 2060, the shuttle loop 2062, and/or the sliding tail 2022 (e.g., due to the tapered cross-sectional thicknesses, etc.).

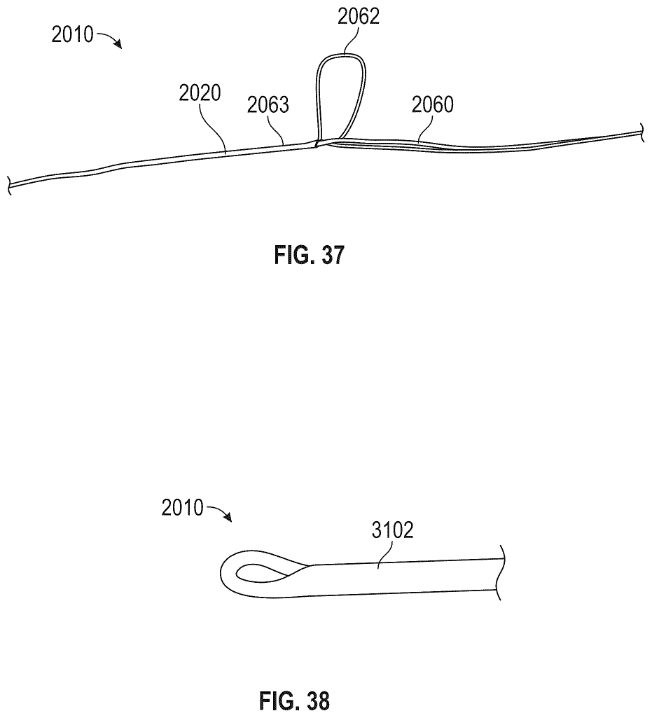

As shown in FIGS. 36-37, once the shuttle 2060 is formed, the shuttle 2060 and/or the shuttle loop 2062 may be passed into, through, or within a suture, for example the working suture 2020. For example, and as discussed herein, the shuttle loop 2062 may be passed into or within the working suture 2020. In an example embodiment, the shuttle loop 2062 is passed into or within the working suture 2020 via manipulation (e.g., pulling, etc.) of the first tail 3004, for example via use of an instrument (e.g., as shown in at least FIG. 36). According to an exemplary embodiment, the manipulation of the first tail 3004 into and/or within the working suture 2020 thereby forms the tucked shuttle loop 2063 within the working suture 2020. In some embodiments, a portion of the shuttle loop 2062 is external to the working suture 2020 (e.g., as shown in FIGS. 36-37). For example, and as shown in FIG. 37, a loop portion of the shuttle loop 2062 is external to the working suture 2020, while the tucked shuttle loop 2063 is positioned within the working suture 20202. In other embodiments, the shuttle loop 2062 is internal to the working suture 2020 (e.g., forming the tucked shuttle loop 2063), and the shuttle loop 2062 is formed by manipulating the tucked shuttle loop 2063 relative to the working suture 2020 (e.g., pulling the tucked shuttle loop 2063 from the working suture 2020, etc.).

Referring now to FIGS. 38-40, components of the suture construct 2010 are shown in greater detail, according to example embodiments. In an example embodiment, FIGS. 38-40 illustrate an example method or process of forming the tucked suture 2030. As described herein, the tucked suture 2030 may include the tucked loop 2032 and the tucked tail 2034, which is secured within the working suture 2020 (e.g., the second hollow portion 2026, etc.), for example via the splice 2035. In an exemplary embodiment, the tucked suture 2030 (e.g., the tucked loop 2032, the tucked tail 2034, etc.) is formed of a size 2-0 suture; however, it should be understood that it is contemplated that in other embodiments the tucked suture 2030 and/or components thereof may be or include any other suitable size sutures, and/or combinations thereof.

In an exemplary embodiment, the tucked suture 2030 is formed using a first suture portion or piece, shown as suture 3102. In an example embodiment, the suture 3102 is a size 2-0 suture. In an exemplary embodiment, the tucked loop 2032 is formed by piercing an opening or aperture in the suture 3102, and passing a first end (e.g., a long end or long tail, etc.) of the suture 3102 through the opening to form a loop. In an exemplary embodiment, the tucked loop 2032 is formed by further piercing an opening in the first end (e.g., long end or long tail, etc.), for example coaxially and/or down a center. Further, the tucked loop 2032 is formed by adjusting the loop to a predetermined or desired size (e.g., diameter, shape, etc.), and then tucking a second end (e.g., short end or short tail) of the suture 3102 into the opening and down the center of the first end, thereby forming the tucked loop 2032 (e.g., as shown in at least FIG. 38, also referred to as an eye splice).

In some embodiments, the tucked loop 2032 is formed by piercing an opening or aperture in the suture 3102, and passing a first end (e.g., a long end or long tail, etc.) of the suture 3102 through the opening to form a loop. In an exemplary embodiment, the tucked loop 2032 is formed by further adjusting the loop to a predetermined or desired size (e.g., diameter, shape, etc.), and piercing an opening in the first end (e.g., long end or tail end, etc.), for example coaxially and/or down a center. Further, the tucked loop 2032 is formed by adjusting (e.g., manipulating, pulling, etc.) the first end (e.g., long end or long tail, etc.) to set the size of the loop, and then tucking a second end (e.g., short end or short tail) of the suture 3102 into the opening and down the center of the first end, thereby forming the tucked loop 2032 (e.g., again, as shown in at least FIG. 38). It should be understood that it is contemplated that the tucked loop 2032 may be formed using any suitable technique, for example for creating a loop (e.g., a fixed-sized loop, a slip loop, a loop that constricts, etc.).

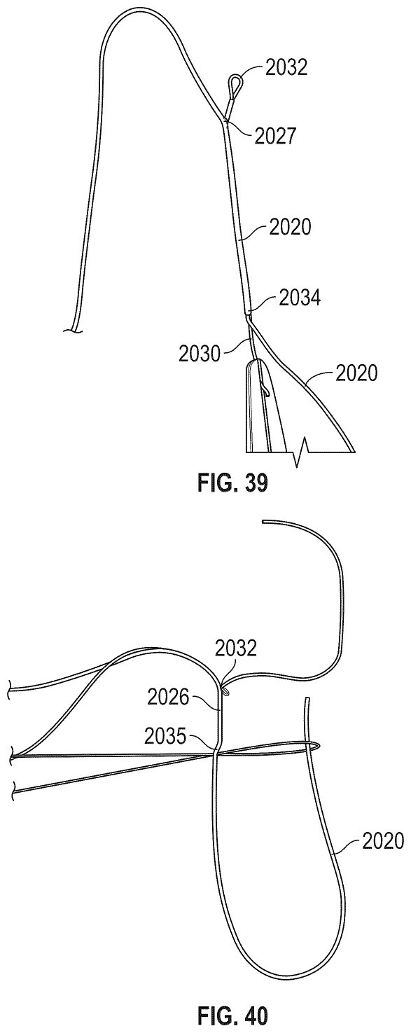

Referring now to FIGS. 39-40, with the tucked loop 2032 of the tucked suture 2030 formed, the tucked suture 2030 may be passed into, through, or within a suture, for example the working suture 2020. For example, and as discussed herein, the tucked suture 2030 may be passed into or within the working suture 2020 (e.g., the second hollow portion 2026, etc.), for example via the opening 2027. In an example embodiment, the tucked suture 2030 is passed into or within the working suture 2020 via manipulation (e.g., pulling, etc.) of an end of the tucked suture 2030 (e.g., as shown in at least FIG. 39). For example, the tucked suture 2030 may be manipulated such that the tucked tail 2034 is positioned within the working suture 2020, as discussed herein. Further, the tucked suture 2030 may be manipulated (e.g., pulled, repositioned, etc.), until or such that the tucked loop 2032 is positioned at the opening 2027 (e.g., at an opening of the second hollow portion 2026, an exit of the first hollow portion 2024, etc.), as discussed herein.

As described herein, with the tucked suture 2030 positioned relative to the working suture 2020, the tucked suture 2303 may be secured relative to the working suture 2020. For example, with the tucked tail 2034 positioned within the second hollow portion 2026, a splice (e.g., the splice 2035) may be formed in the working suture 2020 and/or the tucked suture 2030 (e.g., the tucked tail 2034, etc.). An end of the working suture 2020 may then be passed into and/or through the splice 2035 (e.g., as shown in at least FIG. 40), thereby securing the tucked suture 2030 (e.g., the tucked tail 2034) relative to the working suture 2020 (e.g., via the splice 2035, a self-splice, etc.). It should be understood that in other embodiments, the tucked suture 2030 may be otherwise secured relative to the working suture 2020 using any other suitable technique, according to various embodiments.

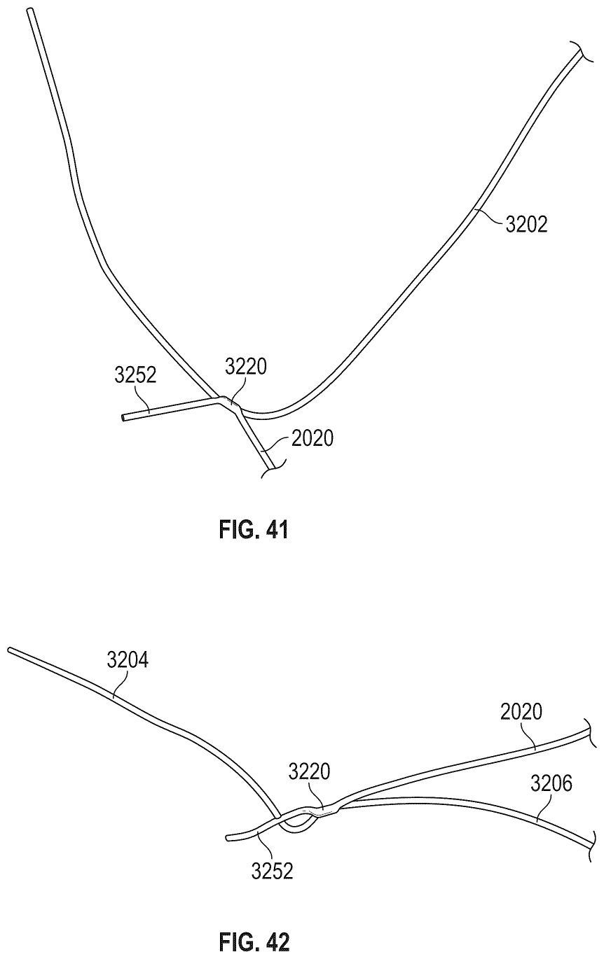

Referring now to FIGS. 41-44, one or more example components of the suture construct 2010 are shown in greater detail, according to example embodiments. In an example embodiment, FIGS. 41-44 illustrate example attachments or components, which may be used in combination with the suture construct 2010. For example, the exemplary features of FIGS. 41-44 may be removably coupled with ends of a suture of the suture construct 2010 (e.g., the fixed tail 2021 and/or the sliding tail 2022 of the working suture 2020, etc.), for example to aid in loading the suture construct 2010 into a suture passer and/or deploying the suture construct 2010. In some embodiments, the suture construct 2010 may be used along with one or more features described with reference to FIGS. 41-44; however, in other embodiments the suture construct 2010 may be used without one or more components described with reference to FIGS. 41-44.

As shown in FIGS. 41-42, a first suture may be provided. In an example embodiment, the first suture is the working suture 2020 of the suture construct 2010, as described with reference to FIGS. 27-37. As described herein, the working suture 2020 may be a size 0 suture. Further, a second suture, shown as second suture 3202, may be provided. The second suture 3202 may be a size 2 suture; however, in other embodiments the second suture 3202 may be another suitable size described herein (e.g., size 1, 3, etc. suture, etc.). According to an exemplary embodiment, and as described herein, the second suture 3202 may be removably coupled with an end of the working suture 2020 (e.g., the fixed tail 2021 and/or the sliding tail 2022, etc.), for example to aid in loading the suture construct 2010 into a suture passer and/or deploying the suture construct 2010 (e.g., to provide for grasping, grabbing, moving, manipulating, etc. one or more components of the suture construct, etc.). In an exemplary embodiment, one or more characteristics of the second suture 3202 (e.g., a size 2 suture, a thicker suture, a stiffer suture, etc.) are such that the second suture 3202 aids in loading the suture construct 2010 into a suture passer and/or deploying the suture construct 2010.

As shown in FIG. 41, the second suture 3202 is passed into, through, or within the working suture 2020. For example, the second suture 3202 may be passed into or through the working suture 2020 at an end of the working suture 2020 (e.g., the fixed tail 2021, the sliding tail 2022), such that the working suture 2020 forms or defines a tail, shown as tail region 3252. Further, due to the configuration of the second suture 3202 and/or the working suture 2020 (e.g., relative sizes of the sutures, etc.), a fixation region, or knotless fixation region, shown as finger trap region 3220, is provided within the working suture 2020 (e.g., due to a coaxial alignment of the second suture 3202 within the working suture 2020, etc.). As described elsewhere herein, the working suture 2020 and/or the second suture 3202 may provide a region (e.g., the finger trap region 3220, etc.) that provides knotless fixation (e.g., via a structural, material, and/or configuration of a hollow portion of the suture(s), etc.), and/or the sutures may provide a region that offers knotless fixation via engagement between one or more components of the suture construct 2010, as described herein.

In an exemplary embodiment, an opening, aperture, hole, or splice may be provided in the tail region 3252. As shown in FIG. 42, an end, shown as a first end 3204, of the second suture 3202 may be passed into and/or through the opening in the tail region 3252. In an example embodiment, the second suture 3202 passes into and/or through the tail region 3252 a distance from the finger trap region 3220 (e.g., 2, 3, 5, etc. millimeters). According to an exemplary embodiment, with the second suture 3202 passed through the working suture 2020 to form the finger trap region 3220, and/or through the opening in the tail region 3252, the second suture 3202 may be removably coupled with the working suture 2020. For example, manipulation (e.g., pulling, application of force, etc.) of an end of the second suture 3202 (e.g., a second end 3206 opposite the first end 3204) may result in a coupling or tightening of the second suture 3202 to the working suture 2020. Conversely, a manipulation (e.g., pulling, application of force, etc.) of an opposite end of the second suture 3202 (e.g., the first end 3204, etc.) would result in a de-coupling or loosening of the second suture 3202 from the working suture 2020. In this regard, the exemplary features shown and described in FIGS. 41-42 provide a suture attachment (e.g., the second suture 3202, etc.), which can be attached to the working suture 2020 of the suture construct 2010 to aid in loading and/or deploying the suture construct 2010, while also detached or de-coupled from the suture construct 2010 (e.g., the working suture 2020, etc.) following deployment.

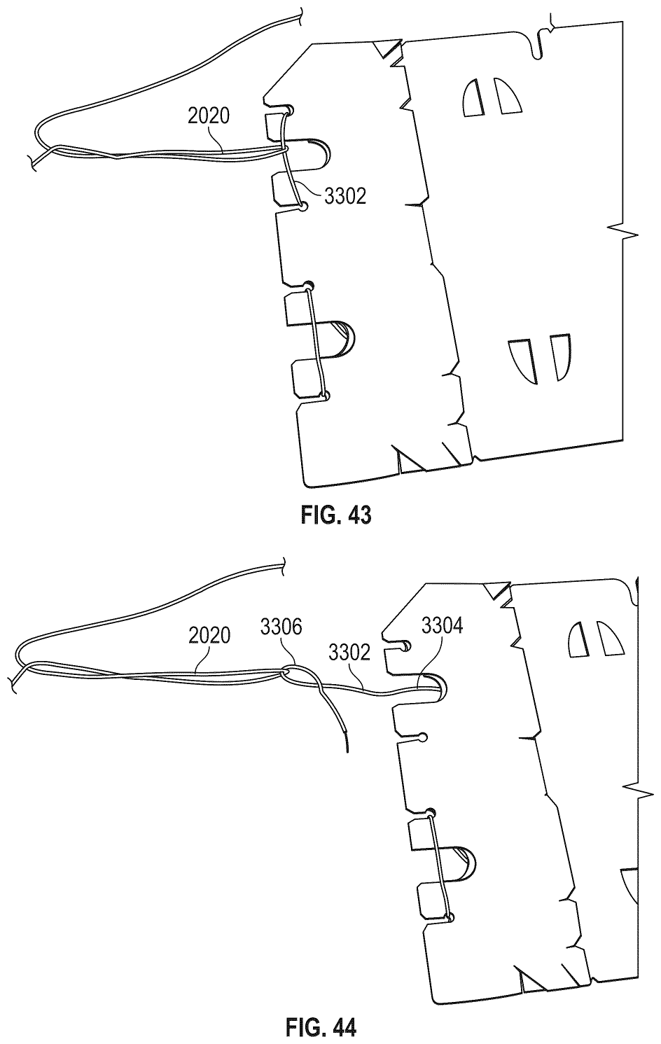

As shown in FIGS. 43-44, an attachment or loop attachment is shown, according to an exemplary embodiment. In some embodiments, one or more components of the attachment shown in FIGS. 43-44 is/are provided in a kit. As shown, a first suture may be provided. In an example embodiment, the first suture is the working suture 2020 of the suture construct 2010, as described with reference to FIGS. 27-37. As described herein, the working suture 2020 may be a size 0 suture. Further, a second suture, shown as second suture 3302, may be provided. In some embodiments, the second suture 3302 a size 2 suture; however, in other embodiments the second suture 3302 may be another suitable size described herein (e.g., size 1, 3, etc. suture, etc.). According to an exemplary embodiment, and as described herein, the second suture 3302 may be removably coupled with an end of the working suture 2020 (e.g., the fixed tail 2021 and/or the sliding tail 2022, etc.), for example to aid in loading the suture construct 2010 into a suture passer and/or deploying the suture construct 2010 (e.g., to provide for grasping, grabbing, moving, manipulating, etc. one or more components of the suture construct, etc.).

As shown in at least FIG. 43, the second suture 3302 is provided in a loop or loop attachment construct. For example, the second suture 3302 is shown to be provided on a card or support, for example in a looped or knotted configuration. In an exemplary embodiment, the looped or knotted configuration is configured to provide a snare configuration; however, in other embodiments the second suture 3302 is otherwise configured. As shown in FIG. 43, the working suture 2020 may be looped over or around (e.g., under, etc.) the second suture 3302, and may be manipulated (e.g., pulled, etc.) to pull the second suture 3302 from the card or support.

As shown in at least FIG. 44, as the working suture 2020 is manipulated, a looped, knotted, or snared configuration is provided between the second suture 3302 and the working suture 2020, thereby removably coupling the second suture 3302 and the working suture 2020. For example, in an exemplary embodiment the working suture 2020 may be looped over or around (e.g., under, etc.) the second suture 3302 (e.g., as shown in at least FIG. 43), and passed into or through a hole, opening, aperture, piercing, or splice in the working suture 2020. In an exemplary embodiment, manipulation (e.g., pulling, application of force, etc.) of a first end of the working suture 2020 results in a coupling, tightening, or snaring of the working suture 2020 to the second suture 3302 (e.g., as shown in at least FIG. 44). Conversely, manipulation (e.g., pulling application of force, etc.) of a second end of the working suture 2020 (e.g., an end opposite the first end, etc.) results in a de-coupling or loosening of the working suture 2020 from the second suture 3302.

Further, in another exemplary embodiment, the second suture 3302 may be provided on a card or support with at least a portion (e.g., an end, a short end, a short tail, etc.) of the second suture 3302 passed into or through a hole, opening, aperture, piercing, or splice in the second suture 3302. In this sense, the second suture 3302 may be passed into or through itself, to form a loop on the card or support (e.g., as shown in at least FIG. 43). In an exemplary embodiment, another portion of the second suture 3302 (e.g., an end, a long end, a long tail, etc.) is further looped around a base or body of the support. As shown, the working suture 2020 may be looped over or around (e.g., under, etc.) the loop formed by the second suture 3302 (e.g., as shown in at least FIG. 43), and manipulation (e.g., pulling, application of force, etc.) of the working suture 2020 results in a coupling, tightening, or snaring of the second suture 3302 to the working suture 2020 (e.g., as shown in at least FIG. 44). In some embodiments, manipulation (e.g., pulling application of force, etc.) of an end of the second suture 3302 (e.g., a short end, a short tail, etc.) results in a de-coupling or loosening of the second suture 3302 from the working suture 2020.

As described with reference to the attachment of FIGS. 41-42, the attachment of FIGS. 43-44 (e.g., the second suture 3302, etc.) may be such that the second suture 3302 is configured to removably couple the working suture 2020. For example, manipulation (e.g., pulling, application of force, etc.) of a first end of the second suture 3302 (e.g., a first end 3304, etc.) may result in a coupling or tightening of the second suture 3302 to the working suture 2020. Conversely, a manipulation (e.g., pulling, application of force, etc.) of a second or an opposite end of the second suture 3302 (e.g., a second end 3306, etc.) would result in a de-coupling or loosening of the second suture 3302 from the working suture 2020. In this regard, the exemplary features shown and described in FIGS. 43-44 provide a suture attachment (e.g., the second suture 3302, etc.), which can be attached to the working suture 2020 of the suture construct 2010 to aid in loading and/or deploying the suture construct 2010, while also detached or de-coupled from the suture construct 2010 (e.g., the working suture 2020, etc.) following deployment.

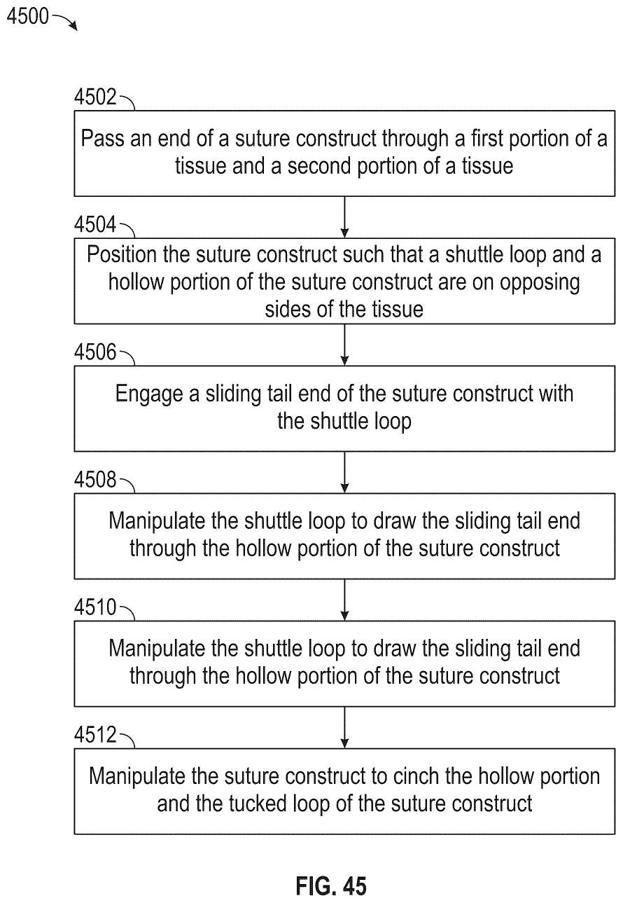

Referring now to FIG. 45, a process, shown as process 4500, for repairing a tissue in a patient is shown, according to an exemplary embodiment. According to an exemplary embodiment, the process 4500 may be implemented using any and/or all of the suture constructs described herein, for example to repair one or more portions of a tissue (e.g., opposing tissue capsules, flaps, or portions, etc.). In particular, the process 4500 is implemented using the suture construct 2010 of FIGS. 27-40 and/or the sutures or attachments of FIGS. 41-44, for example to repair one or more portions of tissue.

Process 4500 is shown to include passing an end of a suture construct through a first portion of a tissue and a second portion of the tissue (4502), according to an exemplary embodiment. In an exemplary embodiment, the process 4500 includes passing the fixed tail 2021 of the working suture 2020 through a first side or portion of the tissue 2800 and out a second or opposing side or portion of the tissue 2800 (e.g., through opposing flaps or portions, etc.). In some embodiments, the process 4500 includes passing the sliding tail 2022 of the working suture 2020 through the tissue 2800 (e.g., through a first side or portion and out a second side or opposite side or portion, etc.). For example, the process 4500 may include passing the sliding tail 2022 of the working suture 2020 through a first side of a first tissue, out a second side of the first tissue, into a first side of a second tissue, and out a second side of the second tissue (e.g., to secure two separate pieces of tissue of a rip, tear, or other tissue damage, etc.). The associated portion of the shuttle 2060 (e.g., as illustrated for example, in FIG. 28) may also be passed through the tissue 2800.

Specifically, process 4500 is shown to include positioning the suture construct to arrange a shuttle loop and a hollow portion of the suture construct on opposing sides of the tissue (4504), according to an exemplary embodiment. In an example embodiment, the process 4500 includes manipulating the fixed tail 2021, the sliding tail 2022, and/or a combination thereof, such that the shuttle loop 2062 and the first hollow portion 2024 are on opposing sides of the tissue 2800. In such a configuration, the shuttle loop 2062 and the shuttle tail 2066 are on opposing sides of the tissue 2800.

In some embodiments, the process 4500 includes slitting, splicing, or dividing a shuttle loop attached or coupled to a suture construct, such as tucked into a portion of the suture construct. For example, the process 4500 may include slitting, splicing, or dividing the tucked shuttle loop 2063 and the working suture 2020. In an example embodiment, dividing the tucked shuttle loop 2063 and the working suture 2020 includes applying a force to a portion of the tucked shuttle loop 2063 (e.g., pulling, etc.), for example to form and/or make the shuttle loop 2062 accessible.

Process 4500 is shown to include engaging a sliding tail end of the suture construct with the shuttle loop (4506), according to an exemplary embodiment. In an exemplary embodiment, the process 4500 includes looping the sliding tail 2022 through the shuttle loop 2062, for example to engage the sliding tail 2022 with the shuttle loop 2062. In some embodiments, the sliding tail 2022 is passed (e.g., looped, drawn, etc.) over or around the tissue 2800 (e.g., the first tissue portion and the second tissue portion, etc.), for example to engage the sliding tail 2022 with the shuttle loop 2062.

Process 4500 is shown to include manipulating the shuttle loop to draw the sliding tail end through the hollow portion of the suture construct (4508), according to an exemplary embodiment. In an exemplary embodiment, with the sliding tail 2022 engaged with the shuttle loop 2062, the shuttle tail 2066 may then be manipulated (e.g., pulled, etc.), thereby moving the shuttle loop 2062 (and the sliding tail 2022). For example, the shuttle tail 2066 may be pulled to move the shuttle loop 2062 (and the sliding tail 2022) through the tissue 2800 (e.g., the first tissue portion, the second tissue portion, etc.), and toward the first hollow portion 2024. In an exemplary embodiment, the manipulation of the shuttle tail 2066 further moves the shuttle loop 2062 (and the sliding tail 2022) into and through the first hollow portion 2024 (e.g., coaxially through, etc.) of the working suture 2020. As described elsewhere herein, first hollow portion 2024 may provide a knotless fixation region. For example, the first hollow portion 2024 receive the sliding tail 2022 to provide a knotless fixation configuration, such as a finger trap or other knotless fixation engagement described elsewhere herein.

Process 4500 is shown to include manipulating the shuttle loop to draw the sliding tail end through a tucked loop of the suture construct (4510), according to an exemplary embodiment. In an exemplary embodiment, the manipulation of the shuttle tail 2066 further moves the shuttle loop 2062 (and the sliding tail 2022) through the tucked loop 2032 of the tucked suture 2030 (e.g., of the working suture 2020, etc.).

Process 4500 is shown to include further manipulating the suture construct to cinch the hollow portion (e.g., the first hollow portion 2024, etc.) and the tucked loop (e.g., the tucked loop 2032, etc.) of the suture (4512), according to an exemplary embodiment. For example, with the shuttle tail 2066 positioned within and/or through the first hollow portion 2024 and the tucked loop 2032, one or more ends of the working suture 2020 may be further manipulated (e.g., tensioned, etc.). For example, the fixed tail 2021 and/or the sliding tail 2022 may be further manipulated (e.g., tensioned, etc.). According to an exemplary embodiment, the tensioning of the fixed tail 2021 and/or the sliding tail 2022 causes the first hollow portion 2024 to provide a knotless fixation region (e.g., the first hollow portion 2024 of the working suture 2020 to cinch onto or engage the sliding tail 2022, etc.) and the tucked loop 2032 to cinch onto the working suture 2020, for example to secure the working suture 2020 (e.g., the suture construct 2010, etc.) in place. According to some embodiments, tensioning the fixed tail 2021 and/or the sliding tail 2022 may cause the first hollow portion 2024 to provide a knotless fixation region, while concurrently (e.g., simultaneously, approximately simultaneously, substantially simultaneously, etc.) causing the tucked loop 2032 to cinch onto the working suture 2020. In some instances, the sliding tail 2022 and/or the fixed tail 2021 may also be manipulated (e.g., pulled, etc.), for example to further position the first hollow portion 2024 (e.g., the knotless fixation region, a finger trap, etc.) relative to the tissue 2800 and/or to tighten the repair of the tissue 2800. At this point, the tear in the tissue 2800 may be closed and/or sealed, for example as part of the repair of the tissue 2800.

In some embodiments, the process 4500 includes removing excess suture construct portions. For example, the process 4500 may include removing (e.g., cutting, splicing, etc.) excess portions of the sliding tail 2022 and/or the fixed tail 2021.

Advantageously, the process 4500 of FIG. 45 allows for deployment of the suture construct 2010 with less force (e.g., shuttling force, tension, etc.) compared to other suture construct configurations, for example where the shuttle loop 2062, the first hollow portion 2024, and/or the provided knotless fixation, such as a finger trap portion, are on the same side of the tissue 2800. For example, and as described herein, in process 4500 the sliding tail 2022 is shuttled through the tissue 2800 (e.g., via the shuttle loop 2062 and/or the shuttle 2060, etc.), and then through the first hollow portion 2024, for example to provide the knotless fixation region, such as a finger trap portion. Such a process results in less force needed to shuttle the sliding tail 2022 (e.g., through the tissue 2800 and then through the first hollow portion 2024, etc.) compared to other processes, which may involve shuttling the sliding tail 2022 first through the first hollow portion 2024 (e.g., which may provide the knotless fixation region, such as a finger trap portion, etc.), and then through the tissue 2800.

Further, by positioning the shuttle loop 2062 and the first hollow portion 2024 on opposing sides of the tissue 2800, the process 4500 of FIG. 45 allows for more control over providing and/or the positioning of the first hollow portion 2024 (e.g., knotless fixation region, such as a finger trap, etc.). For example, the process 4500 of FIG. 45 allows for manipulation of the fixed tail 2021 (e.g., with is on an opposing side of the tissue 2800, etc.) to pull an opening or entrance of the first hollow portion 2024 back toward the tissue 2800, for example without first setting the knotless fixation region, such as a finger trap, within the first hollow portion 2024, which may be done in other processes. For instance, if the first hollow portion 2400 migrates far from the tissue during shuttling (e.g., the shuttling is performed outside of the patient and outside the cannula as in FIGS. 29-32), the fixed tail 2021 may be tensioned to pull the first hollow portion 2024 back towards the tissue. In some instances, the process 4500 of FIG. 45 also provides for less tanging at the first hollow portion 2024, for example by allowing the sliding tail 2022 and/or the first hollow portion 2024 to be pulled away from the tissue 2800 during shuttling, compared to other techniques that may pull these features toward and/or through the tissue 2800 during shuttling, which in some instances may result in tangling or snagging during shuttling. Yet further, in some instances the process 4500 of FIG. 45 may also allow for shuttling the sliding tail 2022 through the first hollow portion 2024 to occur outside a cannula and/or away from the tissue 2800, which results in less force needed for shuttling, improved control over positioning of the first hollow portion 2024, an easier procedure for the operator, and/or reduced tangling as a result of shuttling or deployment. In yet other embodiments, the process 4500 of FIG. 45 also provides a configuration such that if tangling or snaring of one or more of the structures of the suture construct occur, it occurs on a side of the suture construct with the fixed tail (e.g., the fixed tail 2021) rather than the sliding tail (e.g., the sliding tail 2022), which allows the sliding tail 2022 to still be manipulated (e.g., move, etc.) to position the first hollow portion 2024 and/or to provide a knotless fixation region at the first hollow portion 2024, as described herein.

As described herein, the present disclosure relates to one or more kits for preparing a closure of soft tissue. In this regard, it is contemplated that one or more components of the suture construct 2010 may be provided as a kit. For example, a kit may include a working suture (e.g., the working suture 2020, etc.), a shuttle (e.g., the shuttle 2060, etc.), and/or a tucked suture (e.g., the tucked suture 2030). Further, the kit may include one or more attachments or attachment sutures, for example the sutures and/or attachment sutures described with reference to FIGS. 41-44. In some embodiments, the kit includes additional components (e.g., a needle, packaging, a series of packages and/or suture constructs, one or more instructions for use, etc.) described elsewhere herein. All such combinations and embodiments are contemplated herein.

It is to be understood that the disclosure set forth herein includes any possible combinations of the particular features set forth above, whether specifically disclosed herein or not. For example, where a particular feature is disclosed in the context of a particular aspect, embodiment, configuration, or arrangement, that feature may also be used, to the extent possible, in combination with and/or in the context of other particular aspects, embodiments, configurations, and arrangements of the contemplated apparatuses, systems, kits and methods. Non-limiting examples of the aforementioned features include finger traps, tucked loop sutures, eyelets, suture size and suture material.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

As utilized herein with respect to numerical ranges, the terms “approximately,” “about,” “substantially,” and similar terms generally mean+/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. For example, the concepts and/or features of the suture construct of the exemplary embodiment described with reference to at least FIGS. 27-44 may be incorporated in the suture construction of the exemplary embodiment described with reference to FIGS. 1-26. Although only one example of an element from one embodiment that can be incorporated or utilized in another embodiment has been described above, it should be appreciated that other elements of the various embodiments may be incorporated or utilized with any of the other embodiments disclosed herein.

What is claimed:

1. A pre-deployment suture construct comprising:
a working suture including a fixed tail at a first end and a sliding tail at a second end opposite from the first end, wherein the working suture defines a first hollow portion, a second hollow portion, a first entry opening, an exit opening, and a second entry opening, wherein the first hollow portion extends from the first entry opening to the exit opening, and the second hollow portion is positioned closer to the sliding tail than the first hollow portion;
a tucked suture including a tucked loop and a tucked tail extending from the tucked loop, wherein at least a portion of the tucked tail is disposed within the second hollow portion of the working suture via the second opening; and
a shuttle extending through the first hollow portion and the tucked loop,
wherein the sliding tail is configured to be shuttled through the first hollow portion and the tucked loop via the shuttle, wherein the first hollow portion is configured to provide a finger trap with the sliding tail shuttled through the first hollow portion and the tucked loop.

2. The suture construct of claim 1, wherein the shuttle further includes a shuttle loop and a shuttle tail, the shuttle tail extending through the first hollow portion, from the exit opening, and through the tucked loop.

3. The suture construct of claim 2, wherein the shuttle loop and the shuttle tail are positioned on opposing sides of the first hollow portion.

4. The suture construct of claim 2, wherein the shuttle loop includes a tucked shuttle loop portion, the tucked shuttle loop portion positioned within the working suture.

5. The suture construct of claim 4, wherein between the tucked shuttle loop portion and the first hollow portion, the shuttle is positioned externally of the working suture.

6. The suture construct of claim 1, wherein the shuttle further includes a shuttle loop and a shuttle body extending from the shuttle loop, wherein at least a portion of the shuttle loop and the shuttle body being formed of sutures having different diameters.

7. The suture construct of claim 1, wherein the tucked suture is secured within the second hollow portion of the suture via a splice a distance from the tucked loop.

8. The suture construct of claim 7, wherein the distance is such that when the suture construct is deployed, the sliding tail and at least a portion of the tucked suture within the second hollow portion of the suture pass through the first hollow portion and the tucked loop.

9. The suture construct of claim 1, wherein a distance between a tip of the fixed tail and a tip of the sliding tail is at least 45 inches, and a distance between the tip of the fixed tail and the first hollow portion is at least 25 inches.

10. The suture construct of claim 1, wherein the tucked loop is at least partially external to the working suture.

11. The suture construct of claim 1, wherein the tucked loop is external to the working suture.

12. The suture construct of claim 1, wherein with the sliding tail shuttled through the first hollow portion and the tucked loop the tucked loop is configured to secure the sliding tail.

13. A method for securing tissue with a suture construct, the method comprising:
passing one of a fixed tail and a sliding tail of a working suture of a suture construct through or around a tissue, the working suture having a length extending from the fixed tail to the sliding tail;
engaging the sliding tail of the working suture to a first end of a shuttle, the shuttle including a shuttle tail extending from the first end, a first portion of the shuttle tail extending through a first hollow portion of the working suture and a second portion of the shuttle tail separated from the first end by the first portion passing through a tucked loop of a tucked suture, a portion of a tucked tail of the tucked suture extending through a second hollow portion of the working suture, the second hollow portion being further from the fixed tail than the first hollow portion; and
pulling a second end of the shuttle opposite the first end of the shuttle to bring the sliding tail of the working suture through the first hollow portion of the working suture and through the tucked loop of the tucked suture,
wherein once the sliding tail is sufficiently tensioned through the pulling step, a knotless fixation region is provided at the first hollow portion and the tucked loop is cinched.

14. The method of claim 13, further comprising, prior to the passing step, providing the suture construct that includes the working suture, the tucked suture, and the shuttle.

15. The method of claim 13, wherein the tissue is one of a hip capsule, abductors of the hip, subscapularis of a shoulder, rotator cuff of the shoulder, biceps of the shoulder and a meniscus in a knee.

16. The method of claim 13, wherein the passing step includes passing one of the fixed tail and the sliding tail of the working suture around the tissue such that when the knotless fixation region is provided, fracture surfaces of the tissue are opposed to support a bone fracture, the tissue including the fracture surfaces.

17. The method of claim 13, wherein the passing step includes positioning the first end of the shuttle and a portion of the tucked tail within the second hollow of the working suture on opposing sides of the tissue.

18. A kit for securing tissue, the kit comprising:
a working suture including a fixed tail at a first end and a sliding tail at a second end opposite from the first end, wherein the working suture defines a first hollow portion, a second hollow portion, a first entry opening, an exit opening, and a second entry opening, wherein the first hollow portion extends from the first entry opening to the exit opening, and the second hollow portion is positioned closer to the sliding tail than the first hollow portion;
a tucked suture including a tucked loop and a tucked tail extending from the tucked loop, wherein at least a portion of the tucked tail is disposed within the second hollow portion of the working suture via the second opening;
a shuttle extending through the first hollow portion and the tucked loop; and
a suture attachment, the suture attachment configured to couple at least one of the fixed tail and the sliding tail in a first, pre-deployment configuration and de-couple the at least one of the fixed tail and the sliding tail in a second, deployment configuration,
wherein the sliding tail is configured to be shuttled through the first hollow portion and the tucked loop via the shuttle, wherein the first hollow portion is configured to provide a finger trap with the sliding tail shuttled through the first hollow portion and the tucked loop.

19. The kit of claim 18, wherein the shuttle further includes a shuttle loop and a shuttle body extending from the shuttle loop, the shuttle loop and the shuttle body being formed of sutures having different diameters.

20. The kit of claim 18, wherein a distance between an end of the fixed tail and an end of the sliding tail is at least 45 inches, and a distance between the end of the fixed tail and the first hollow portion is at least 25 inches.
